SEQUENCE LISTING (8 sequences)

SEQ ID NO: 1 - protein, 122 aa, Artificial Sequence
  felinized anti-NGF antibody VH Sequence
QVQLMESGADLVQPSESLRLTCVASGLSLTNNNVNWVRQAPGKGLEWMGGVWAGGATDYNSALKSRLTITRDTSKNTVFLQMHSLQSEDTATYYCARDGGYSSSTLYAMDAWGQGTTVTVSA

SEQ ID NO: 2 - protein, 107 aa, Artificial Sequence
  felinized anti-NGF antibody VL Sequence
DIEMTQSPLSLSATPGETVSISCRASEDIYNALAWYLQKPGRSPRLLIYNTDTLHTGVPDRFSGSGSGTDFTLKISRVQTEDVGVYFCQHYFHYPRTFGQGTKLELK

SEQ ID NO: 3 - protein, 476 aa, Artificial Sequence
  felinized anti-NGF antibody HC Sequence
MEWSWVFLFFLSVTTGVHSQVQLMESGADLVQPSESLRLTCVASGLSLTNNNVNWVRQAPGKGLEWMGGVWAGGATDYNSALKSRLTITRDTSKNTVFLQMHSLQSEDTATYYCARDGGYSSSTLYAMDAWGQGTTVTVSAASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 4 - protein, 237 aa, Artificial Sequence
  felinized anti-NGF antibody LC Sequence
MSVPTQVLGLLLLWLTDARCDIEMTQSPLSLSATPGETVSISCRASEDIYNALAWYLQKPGRSPRLLIYNTDTLHTGVPDRFSGSGSGTDFTLKISRVQTEDVGVYFCQHYFHYPRTFGQGTKLELKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 5 - protein, 122 aa, Artificial Sequence
  felinized anti-NGF antibody (V5_1) VH Sequence
QVQLVESGGDLVQPGGSLRLTCVASGFSLTNNNVNWVRQAPGKGLEWMGGVWAGGATDYNSAVKSRLTITRDTSKNTVFLQMHSLQSEDTATYYCARDGGYSSSTLYAMDAWGQGTTVTVSA

SEQ ID NO: 6 - protein, 122 aa, Artificial Sequence
  felinized anti-NGF antibody (V62) VH Sequence
QVQLVESGGDLVQPGGSLRLTCVASGFSLTNNNVNWVRQAPGKGLEWMGGVWAGGATDYNSALKSRLTITRDTSKNTVFLQMHSLQSEDTATYYCARDGGYSSSTLYAMDVWGQGTTVTVSA

SEQ ID NO: 7 - protein, 122 aa, Artificial Sequence
  felinized anti-NGF antibody (V73) VH Sequence
QVQLVESGGDLVQPGGSLRLTCVASGFSLTNNNVNWVRQAPGKGLEWMGGVWAGGATDYNSALKSRLTITRDTSKNTVFLQMHSLQSEDTATYYCARDGGYSSSTLYAMEAWGQGTTVTVSA

SEQ ID NO: 8 - protein, 457 aa, Artificial Sequence
  felinized anti-NGF antibody (V5_1) HC Sequence
QVQLVESGGDLVQPGGSLRLTCVASGFSLTNNNVNWVRQAPGKGLEWMGGVWAGGATDYNSAVKSRLTITRDTSKNTVFLQMHSLQSEDTATYYCARDGGYSSSTLYAMDAWGQGTTVTVSAASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

(19) United States
(12) Patent Application Publication
(10) Pub. No.: US 2023/0391862 A1
(43) Pub. Date: Dec. 7, 2023

(54) METHOD OF SUPPRESSING IMMUNE RESPONSE TO VECTOR-DELIVERED THERAPEUTIC PROTEIN

(71) Applicant: Scout Bio, Inc., Philadelphia, PA (US)

(72) Inventors: Samantha J. BUSFIELD, Victoria (AU); Matthew J. WILSON, Philadelphia, PA (US)

(21) Appl. No.: 18/033,279

(22) PCT Filed: Oct. 20, 2021
(86) PCT No.: PCT/US2021/055881

§ 371 Date: Apr. 21, 2023

Related U.S. Application Data
(60) Provisional application No. 63/104,370, filed on Oct. 22, 2020.

(51) Int. Cl. C07K 16/22; A61P 19/02; C07K 16/24; C12N 15/86

(52) U.S. Cl. CPC C07K 16/22; A61P 19/02; A61K 2039/552; C12N 15/86; C07K 16/244

(57) ABSTRACT

Provided are methods for suppressing an immune response to a therapeutic protein encoded by a vector in a subject in need thereof and associated compositions. The method may include selecting a subject as the subject in need of treatment with the vector and then administering to the subject a composition comprising the therapeutic protein or a homolog or functional variant thereof, optionally an immunologically related variant thereof.

### TECHNICAL FIELD

This disclosure relates generally to vectors comprising polynucleotides for use in gene therapy, gene editing, or other applications.

### STATEMENT REGARDING THE SEQUENCE LISTING

The Sequence Listing associated with this application is provided in text format in lieu of a paper copy and is hereby incorporated by reference into the specification. The name of the text file containing the Sequence Listing is SCTB_010_01WO_ST25.txt. The text file is about 20 KB, created on Oct. 18, 2021, and is being submitted electronically via EFS-Web.

### BACKGROUND

Various of types of vectors, including but not limited to viral vectors, may be used to deliver polynucleotide sequences of interest to the cells of a subject in vivo. When a subject is administered such a vector, the subject may develop an immune response to the therapeutic protein encoded by the vector. This immune response may decrease the efficacy of the vector. There is a need in the art for compositions and methods related to suppression of the immune response to a therapeutic protein encoded by the vector and/or treating a subject with a vector.

### SUMMARY OF THE INVENTION

One aspect of the present disclosure relates to a method for suppressing an immune response to a vector comprising a polynucleotide encoding a therapeutic protein in a subject in need thereof, for expressing a therapeutic protein in a subject in need thereof, and/or for treating a disease or disorder associated with reduced expression of a therapeutic protein in a subject in need thereof, the method comprising: a) selecting a subject as the subject in need of treatment with the vector, and then b) administering to the subject a composition comprising the therapeutic protein or a homolog or functional variant thereof, optionally an immunologically related variant thereof.

In some embodiments, the method comprises administering to the subject the vector comprising the polynucleotide encoding the therapeutic protein.

In some embodiments, the method comprises administering to the subject the composition comprising the therapeutic protein, and administering to the subject the vector comprising the polynucleotide encoding the therapeutic protein.

In some embodiments, the therapeutic protein is administered before the vector is administered.

In some embodiments, the therapeutic protein is administered after the vector is administered.

In some embodiments, the therapeutic protein is administered at about the same time the vector is administered.

In some embodiments, the therapeutic protein administered is the same as the therapeutic protein encoded by the vector.

In some embodiments, the therapeutic protein administered is similar but not identical to the therapeutic protein encoded by the vector.

In some embodiments, the vector is a viral vector.

In some embodiments, the viral vector is an adeno-associated virus (AAV) vector.

In some embodiments, the AAV vector is an AAV1 vector.

In some embodiments, the vector is administered at a dose of 1×10⁷ to 1×10¹⁵ gc/kg.

In some embodiments, the vector is administered at a dose of 1×10⁹ to 1×10¹³ gc/kg.

In some embodiments, the vector is administered at a dose of 2.5×10¹¹ gc/kg.

In some embodiments, the therapeutic protein is administered intramuscularly.

In some embodiments, the therapeutic protein is administered intravenously.

In some embodiments, the therapeutic protein or a functional fragment of the therapeutic protein shares at least 95% identity to an endogenous protein of the subject or a functional fragment of the endogenous protein.

In some embodiments, the therapeutic protein is recognized by the immune system as a self-antigen.

In some embodiments, the composition is administered to the subject at a dose effective to suppress an immune response to the therapeutic protein encoded by the vector.

In some embodiments, the therapeutic protein comprises an antibody or antigen-binding fragment thereof.

In some embodiments, the therapeutic protein is a monoclonal antibody.

In some embodiments, the monoclonal antibody is a monoclonal antibody that specifically binds Nerve Growth Factor (NGF).

In some embodiments, the monoclonal antibody is a monoclonal antibody that specifically binds erythropoietin (EPO), IL-31, vascular endothelial growth factor (VEGF), CD20, human epidermal growth factor receptor 2 (Her2), tumor necrosis factor (TNF), immunoglobulin E (IgE), IL-2, IL-33, CD52, CD3, CD19, IL-6, IL-4, IL-4R, IL-4R trap, IL-13, IL-13R, IL-5, IL-5R, IL-5R trap, IL-33R, ST2 Receptor, IL-1RA, α4β7 integrin, IL-12, IL-23, Granulocyte-macrophage colony-stimulating factor (GM-CSF), GM-CSF Receptor, Programmed cell death protein 1 (PD-1), Programmed death-ligand 1 (PD-L1), cytotoxic T-lymphocyte-associated protein 4 (CTLA-4), B7-1 (CD80), or B7-2 (CD86).

In some embodiments, the therapeutic protein is a bispecific antibody. The bispecific antibody may specifically bind CD3 (a “BITE”). In some embodiments, the therapeutic protein is an antibody-like molecule that binds to an immune effector cell (e.g., T cell, NK Cell) and an antigen of interest, to promote cell killing.

In some embodiments, the therapeutic protein is Glucagon-like peptide 1 (GLP-1), insulin, Gonadotropin-releasing hormone (GnRH), or Müllerian inhibiting substance (MIS)/anti-Müllerian hormone (AMH).

In some embodiments, the therapeutic protein is an agonist or antagonist of GLP-1 insulin, GnRH, or MIS/AMH.

In some embodiments, the subject is a feline.

In some embodiments, the subject is a canine.

In some embodiments, the subject is a human.

In some embodiments, the therapeutic protein is a feline monoclonal antibody or a felinized monoclonal antibody.

In some embodiments, the subject is a juvenile or an adult.

In some embodiments, the therapeutic protein is administered at a dose of less than about 50 mg/kg, less than about 25 mg/kg, less than about 10 mg/kg, less than about 5 mg/kg, less than about 1 mg/kg, or less than about 0.5 mg/kg.

In some embodiments, the therapeutic protein is administered at a dose of at least about 0.01 mg/kg, at least about 0.05 mg.kg, at least about 0.1 mg/kg, at least about 1 mg/kg, at least about 2 mg/kg, at least about 5 mg/kg, at least about 10 mg/kg, or at least about 15 mg/kg.

In some embodiments, the therapeutic protein is administered at a dose of about 0.1 mg/kg to about 50 mg/kg, about 1 mg/kg to about 50 mg/kg, about 0.1 mg/kg to about 25 mg/kg, about 1 mg/kg to about 25 mg/kg, or about 5 mg/kg to about 10 mg/kg.

In some embodiments, the therapeutic protein is administered at a dose of about 0.1 mg/kg, about 1 mg/kg, about 2 mg/kg, about 5 mg/kg, about 10 mg/kg, or about 15 mg/kg.

In some embodiments, the therapeutic protein is administered at a sub-therapeutic dose for the therapeutic protein.

In some embodiments, the therapeutic protein is administered to the subject for less than 6 weeks, less than 5 weeks, less than 4 weeks, less than 3 weeks, less than 2 weeks, less than 1 week, less than 5 days, less than 3 days, or less than one day before the vector is administered.

In some embodiments, the immune response is an antibody-based immune response of the subject to the therapeutic protein in response to expression of the therapeutic protein by cells transduced with the vector in the subject.

In some embodiments, the method reduces the host antibody response by at least about 20%.

In some embodiments, the method reduces the host antibody response by at least about 50%.

In some embodiments, the method reduces the host antibody response by at least about 75%.

In some embodiments, the method reduces the host antibody response by at least about 90%.

In some embodiments, the therapeutic protein is administered 1 day after, 2 days after, 5 days after, 7 days after, 10 days after, or 20 days after the vector is administered.

In some embodiments, the subject is treatment-naïve to the therapeutic protein.

In some embodiments, the therapeutic protein is administered at a time which would allow measurable levels of the therapeutic protein to be present at the time of administration of the vector.

In some embodiments, the therapeutic protein is administered before expression of the therapeutic protein by the vector would be expected.

Other aspects and advantages of the invention will be readily apparent from the following detailed description of the invention.

### BRIEF DESCRIPTION OF FIGURES

FIG. 1 is a series of diagrams of illustrative embodiments of the methods of the present disclosure. FIG. 1A depicts the administration of a protein before the administration of a vector comprising a polynucleotide. FIG. 1B depicts the administration of a protein slightly before the administration of a vector comprising a polynucleotide. FIG. 1C depicts the administration of a protein on the same day as the administration of a vector comprising a polynucleotide. FIG. 1D depicts the administration of a protein slightly after the administration of a vector comprising a polynucleotide.

FIG. 2 is a diagram of an illustrative protocol outline depicting the pre-treatment methods and corresponding day of administration, AAV.CB7.feNGF administration at day 0, and the period of time serum antibody levels and anti-transgene product antibody (ATPA) response were monitored.

FIG. 3 is a series of graphs depicting serum levels of feNGF mAb (FIGS. 3A-3C) or ATPA levels (FIGS. 3D-3F) in cats following day 0 intramuscular administration of AAV containing the feNGF mAb transgene under the control of a CB7 promoter at a dose of 1×10¹¹ gc/kg (FIG. 3A, FIG. 3D), 5×10¹¹ gc/kg (FIG. 3B, FIG. 3E), or 3×10¹² gc/kg (FIG. 3C, FIG. 3F). Numbers associated with individual samples denote a subject in the cohort (e.g., FIG. 3A subjects 342, 127, 330, and 129). PK denotes pharmacokinetics and ATPA denotes anti-transgene product antibody.

FIG. 4 is a series of graphs depicting serum levels of feNGF mAb (FIGS. 4A-4D) or ATPA levels (FIGS. 4E-4H) in cats following day 0 intramuscular administration of AAV containing the feNGF mAb transgene under the control of a CB7 promoter at a dose of 2.5×10¹¹ gc/kg. Prednisolone (FIGS. 4A and 4E), recombinant mAb (FIGS. 4B and 4F), AAV8.TBG-feNGF(IV) (FIGS. 4C and 4G), or AAV.TBG-feNGF(IV) (FIGS. 4D and 4H) pre-treatments were administered to the cats according to the protocol timeline of FIG. 2. Numbers associated with individual samples denote a subject in the cohort (e.g., FIG. 4A subjects 802, 807, 818, and 094).

FIG. 5 is a study design diagram. Study participant cats are evaluated at a screening visit (Day −14 to −3) and at visits on Days 0, 14, 30, 60, 90, 120, 150 and 180. The Day 14 visit occurs within ±1 day (i.e. a three-day window, Day 13-15) of the scheduled date, the Day 30 visit and all other visits occur within ±3 days.

FIG. 6 is a study design diagram. The first cohort receives a single intramuscular (IM) dose of an AAV vector containing the transgene encoding the fGLP-1mut-Fc protein (AAV-fGLP-1mut-Fc). The vector is administered at 1×10¹¹ gc/cat on Day 0. The second cohort is given three subcutaneous (SC) doses, at 65 μg/cat, of recombinant purified feGLP-1mut-Fc at Day −14, Day −7 and Day −1 prior to also receiving the same AAV-fGLP-1mut-Fc dose as cohort 1 at Day 0. Plasma levels of fGLP-1mut-Fc and anti-fGLP-1mut-Fc antibody responses are assessed at weekly intervals for the duration of the study.

FIG. 7 is a study design diagram. The first cohort receives a single IM dose of an AAV vector containing the transgene encoding the heavy and light chains of an IL-31 antibody (AAV-caIL-31). This is administered at 5×10¹¹ gc/kg on Day 0. The second cohort is given a 2 mg/kg subcutaneous (SC) dose of recombinant canine anti-IL-31 antibody at Day −14, prior to also receiving the same AAV-caIL-31 dose as cohort 1 at Day Serum levels of anti-IL-31 and anti-(anti caIL-31 antibody) antibody responses are assessed at weekly intervals for the duration of the study.

FIG. 8 is a study design diagram. The first cohort receives a single IM dose of an AAV vector containing the transgene encoding the heavy and light chains of an anti-canine NGF antibody (AAV-caNGF). This is administered at 5×10¹¹ gc/kg on Day 0. The second cohort will be given a 2 mg/kg subcutaneous (SC) dose of recombinant canine anti-NGF antibody at Day −14, prior to also receiving the same AAV-caNGF dose as cohort 1 at Day Serum levels of anti-NGF and anti-(anti-caNGF antibody) antibody responses are assessed at weekly intervals for the duration of the study.

FIG. 9 is a study design diagram. Cat subjects were administered AAV containing a feNGF mAb (feNGFV5_1) transgene IM at a dose of 2.5×10¹¹ gc/kg at Day 0. The subjects received a 2 mg/kg subcutaneous (SC) dose of either clone JCV4 or clone V5_1 recombinant feline anti-NGF antibody at Day −14, prior to also receiving the same AAV-feNGF dose on Day 0.

FIG. 10A is a graph depicting serum feNGF mAb (μg/mL) as a function of Study Day in animals that received no pre-treatment. #816, #819, #095, #093 each represent one animal subject.

FIG. 10B is a graph depicting serum feNGF mAb (μg/mL) as a function of Study Day in animals that received the purified feNGF mAb (clone JCV4) as a pre-treatment. #811, #084, #089 each represent one animal subject.

FIG. 10C is a graph depicting serum feNGF mAb (μg/mL) as a function of Study Day in animals that received the purified feNGF mAb (clone V5_1) as a pre-treatment. #815, #812, #097, #086 each represent one animal subject.

FIG. 10D is a graph depicting ATPA signal [Relative Light Units (RLU)] as a function of Study Day in animals that received no pre-treatment. #816, #819, #095, #093 each represent one animal subject.

FIG. 10E is a graph depicting ATPA signal [Relative Light Units (RLU)] as a function of Study Day in animals that received the purified feNGF mAb (clone JCV4) as a pre-treatment. #811, #084, #089 each represent one animal subject.

FIG. 10F is a graph depicting ATPA signal [Relative Light Units (RLU)] as a function of Study Day in animals that received the purified feNGF mAb (clone V5_1) as a pre-treatment. #815, #812, #097, #086 each represent one animal subject.

FIG. 11 is a study design diagram. Cat subjects were administered AAV containing a feNGF mAb transgene (feNGFV5_1) at two different doses: either 1×10¹² gc/cat or 1×10¹³ gc/cat at Day 0. The subjects received a 2 mg/kg single subcutaneous dose of purified feNGF mAb (clone V5_1), 14 days prior to receiving the AAV-feNGF dose Day 0.

FIG. 12A is a graph depicting serum feNGF mAb (μg/mL) as a function of Study Day in animals that received no pre-treatment prior to being administered an AAV containing the feNGF mAb (clone V5_1) transgene at a dose of 1×10¹² gc/cat. #501, #502, #503, #504, #505, #506, #507, and #508 each represent one animal subject.

FIG. 12B is a graph depicting serum feNGF mAb (μg/mL) as a function of Study Day in animals that received a 2 mg/kg single subcutaneous dose of purified feNGF mAb (clone V5_1), 14 days prior to receiving an AAV containing the feNGF mAb (clone V5_1) transgene at a dose of 1×10¹² gc/cat at Day 0. #416, #417, #418, #419, #420, #421, #422, and #423 each represent one animal subject.

FIG. 12C is a graph depicting serum feNGF mAb (μg/mL) as a function of Study Day in animals that received a 2 mg/kg single subcutaneous dose of purified feNGF mAb (clone V5_1), 14 days prior to receiving an AAV containing the feNGF mAb (clone V5_1) transgene at a dose of 1×10¹³ gc/cat at Day 0. #424, #425, #426, #427, #428, #429, #430, and #431 each represent one animal subject.

FIG. 12D is a graph depicting ATPA signal [Relative Light Units (RLU)] as a function of Study Day in animals that received no pre-treatment prior to being administered AAV containing the feNGF mAb (clone V5_1) transgene at a dose of 1×10¹² gc/cat. #501, #502, #503, #504, #505, #506, #507, and #508 each represent one animal subject.

FIG. 12E is a graph depicting ATPA signal [Relative Light Units (RLU)] as a function of Study Day in animals that received a 2 mg/kg single subcutaneous dose of purified feNGF mAb (clone V5_1), 14 days prior to receiving an AAV containing the feNGF mAb (clone V5_1) transgene at a dose of 1×10¹² gc/cat at Day 0. #416, #417, #418, #419, #420, #421, #422, and #423 each represent one animal subject.

FIG. 12F is a graph depicting ATPA signal [Relative Light Units (RLU)] as a function of Study Day in animals that received a 2 mg/kg single subcutaneous dose of purified feNGF mAb (clone V5_1), 14 days prior to receiving an AAV containing the feNGF mAb (clone V5_1) transgene at a dose of 1×10¹³ gc/cat at Day 0. #424, #425, #426, #427, #428, #429, #430, and #431 each represent one animal subject.

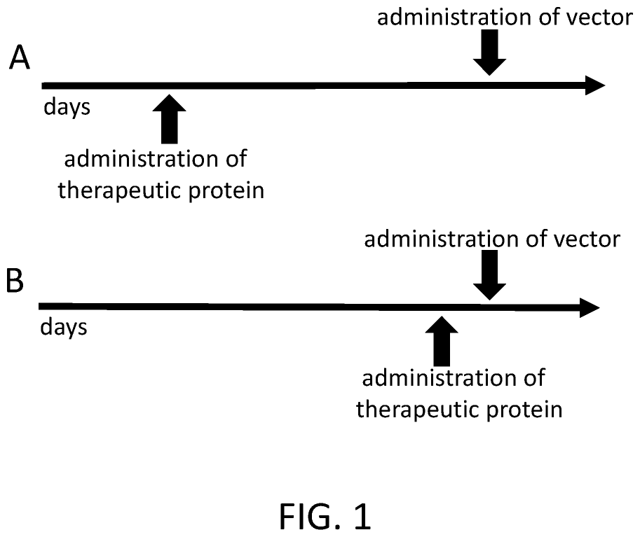

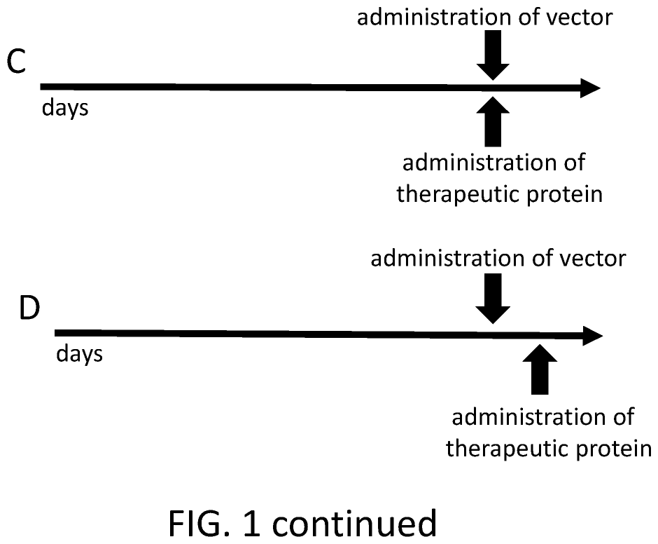

### DETAILED DESCRIPTION OF THE INVENTION

The present disclosure is predicated, at least in part, on the inventors' surprising finding, in administering a vector to express a therapeutic protein, treatment of the subject with the therapeutic protein abrogates the subsequent immune response to the therapeutic protein often observed after the administration of the vector. Advantageously, the inventors found that the abrogated immune response allows for long-term sustained expression of the therapeutic protein and/or improved efficacy.

Accordingly, provided herein are compositions and methods related generally to suppressing an immune response to a vector comprising a polynucleotide encoding a therapeutic protein by administering the same or similar protein before or slightly after the vector.

### Definitions

The terminology used herein is for the purpose of describing particular embodiments only and is not intended to be limiting. Unless defined otherwise, all technical and scientific terms used herein have the same meanings as commonly understood by one of ordinary skill in the art to which the present disclosure belongs. Any materials and methods similar or equivalent to those described herein can be used to practice the present invention. Practitioners may refer to Sambrook et al. (1989) Molecular Cloning: A Laboratory Manual, 2nd ed., Cold Spring Harbor Press, Plainsview, N.Y., and Ausubel et al. (1999) Current Protocols in Molecular Biology (Supplement 47), John Wiley & Sons, New York, Murphy et al. (1995) Virus Taxonomy Springer Verlag: 79-87, for definitions and terms of the art and other methods known to the person skilled in the art.

As used herein, the term “about” refers to a quantity, level, value, dimension, size, or amount that varies by as much as 10% (e.g, by 10%, 9%, 8%, 7%, 6%, 5%, 4%, 3%, 2% or 1%) to a reference quantity, level, value, dimension, size, or amount.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein the singular forms “a”, “an” and “the” include plural aspects unless the context clearly dictates otherwise. For example, reference to “a vector” includes a single vector, as well as two or more vectors; reference to “a cell” includes one cell, as well as two or more cells; and so forth.

### Embodiments

One aspect of the present disclosure relates to a method for suppressing an immune response to a vector comprising a polynucleotide encoding a therapeutic protein in a subject in need thereof, for expressing a therapeutic protein in a subject in need thereof, and/or for treating a disease or disorder associated with reduced expression of a therapeutic protein in a subject in need thereof, the method comprising:
a) selecting a subject as the subject in need of treatment with the vector, and then
b) administering to the subject a composition comprising the therapeutic protein or a homolog or functional variant thereof, optionally an immunologically related variant thereof.

In some embodiments, the method comprises administering to the subject the vector comprising the polynucleotide encoding the therapeutic protein.

In some embodiments, the method comprises administering to the subject the composition comprising the therapeutic protein, and administering to the subject the vector comprising the polynucleotide encoding the therapeutic protein.

In some embodiments, the therapeutic protein is administered before the vector is administered.

In some embodiments, the disease or disorder is degenerative joint disease (DJD).

In some embodiments, the therapeutic protein is administered no more than 1 day, 2 days, 3 days, 5 days, 7 days, 10 days, 12 days, 15 days, 17 days, 21 days, 25 days, 30 days, 35 days, 40 days, 45 days, 50 days, 60 days, 70 days, 80 days, or 90 days before the vector is administered.

In some embodiments, the therapeutic protein is administered at least 1 day, at least 2 days, at least 3 days, at least 5 days, at least 7 days, at least 10 days, at least 12 days, at least 15 days, at least 17 days, at least 21 days, at least 25 days, at least 30 days, at least 35 days, at least 40 days, at least 45 days, at least 50 days, at least 60 days, at least 70 days, at least 80 days, or at least 90 days before the vector is administered.

In some embodiments, the therapeutic protein is administered after the vector is administered.

In some embodiments, the therapeutic protein is administered 1 day after, 2 days after, 5 days after, 7 days after, 10 days after, or 20 days after the vector is administered.

In some embodiments, the therapeutic protein is administered at most 1 day after, at most 2 days after, at most 5 days after, at most 7 days after, at most 10 days after, or at most 20 days after the vector is administered.

In some embodiments, the therapeutic protein is administered at about the same time the vector is administered.

In some embodiments, the vector is a viral vector.

In some embodiments, the viral vector is an adeno-associated virus (AAV) vector.

In some embodiments, the AAV vector is an AAV1 vector.

In some embodiments, the vector is administered at a dose of 1×10⁷ to 1×10¹⁵ gc/kg.

In some embodiments, the vector is administered at a dose of 1×10⁹ to 1×10¹³ gc/kg.

In some embodiments, the vector is administered at a dose of 2.5×10¹¹ gc/kg.

In some embodiments, the vector is administered at a dose of about 1×10⁷ gc/kg, about 1×10⁸ gc/kg, about 1×10⁹ gc/kg, about 1×10¹⁰ gc/kg, about 1×10¹¹ gc/kg, about 1×10¹² gc/kg, about 1×10¹³ gc/kg, about 1×10¹⁴ gc/kg, or about 1×10¹⁵ gc/kg.

In some embodiments, the therapeutically effective dose of vector is about 1×10⁷ gc/kg, about 1×10⁸ gc/kg, about 1×10⁹ gc/kg, about 1×10¹⁰ gc/kg, about 1×10¹¹ gc/kg, about 1×10¹² gc/kg, about 1×10¹³ gc/kg, about 1×10¹⁴ gc/kg, or about 1×10¹⁵ gc/kg.

In an embodiment, the therapeutically effective dose of vector is about 2.5×10¹¹ gc/kg.

In some embodiments, the therapeutically effective dose of vector is about 1×10⁷ gc/kg to about 1×10¹⁰ gc/kg, ab out 1×10⁸ gc/kg to about 1×10¹¹ gc/kg, about 1×10⁹ gc/kg to about 1×10¹² gc/kg, about 1×10¹⁰ gc/kg to about 1×10¹³ gc/kg, about 1×10¹⁵ gc/kg to about 1×10¹⁴ gc/kg, or about 1×10¹² gc/kg to about 1×10¹⁵ gc/kg.

In some embodiments, the vector is administered intramuscularly.

In some embodiments, the vector is administered intravenously.

In some embodiments, the vector is administered subcutaneously.

In some embodiments, the therapeutic protein comprises an antibody or antigen-binding fragment thereof.

In some embodiments, the therapeutic protein is a monoclonal antibody.

In some embodiments, the monoclonal antibody is a monoclonal antibody that specifically binds Nerve Growth Factor (NGF).

In some embodiments, the NGF specifically bound by the monoclonal antibody is human NGF, canine NGF, feline NGF and equine NGF. The present disclosure extends to antigen binding molecules that bind specifically to native NGF (i.e., naturally-occurring NGF), as well as to functional variants thereof. Such functional variants may include NGF molecules that differ from a naturally occurring (wild-type) molecule by one or more amino acid substitutions, deletions and/or insertions. Variant NGF molecules of this type may be naturally-occurring or synthetic (e.g., recombinant) forms.

In some embodiments, the monoclonal antibody that specifically binds NGF shares at least at least 70%, at least 75%, at least 80%, at least 85%, at least 90%, at least 95%, at least 99%, or 100% sequence identity to the felinized anti-NGF antibody of SEQ ID NOs. 1-8.

felinized anti-NGF antibody VH Sequence:
(SEQ ID NO: 1)
QVQLMESGADLVQPSESLRLTCVASGLSLTNNNVNWVRQAPGKGLEWMG
GVWAGGATDYNSALKSRLTITRDTSKNTVFLQMHSLQSEDTATYYCARD
GGYSSSTLYAMDAWGQGTTVTVSA
felinized anti-NGF antibody VL Sequence:
(SEQ ID NO: 2)
DIEMTQSPLSLSATPGETVSISCRASEDIYNALAWYLQKPGRSPRLLIY
NTDTLHTGVPDRFSGSGSGTDFTLKISRVQTEDVGVYFCQHYFHYPRTF
GQGTKLELK
felinized anti-NGF antibody HC Sequence:
(SEQ ID NO: 3)
MEWSWVFLFFLSVTTGVHSQVQLMESGADLVQPSESLRLTCVASGLSLT
NNNVNWVRQAPGKGLEWMGGVWAGGATDYNSALKSRLTITRDTSKNTVF
LQMHSLQSEDTATYYCARDGGYSSSTLYAMDAWGQGTTVTVSAASTTAP
SVFPLAPSCGTTSGATVALACLVLGYFPEPVTVSWNSGALTSGVHTFPA
VLQASGLYSLSSMVTVPSSRWLSDTFTCNVAHPPSNTKVDKTVRKTDHP
PGPKPCDCPKCPPPEMLGGPSIFIFPPKPKDTLSISRTPEVTCLVVDLG
PDDSDVQITWFVDNTQVYTAKTSPREEQFNSTYRVVSVLPILHQDWLKG
KEFKCKVNSKSLPSPIERTISKAKGQPHEPQVYVLPPAQEELSRNKVSV
TCLIKSFHPPDIAVEWEITGQPEPENNYRTTPPQLDSDGTYFVYSKLSV
DRSHWQRGNTYTCSVSHEALHSHHTQKSLTQSPGK
felinized anti-NGF antibody LC Sequence:
(SEQ ID NO: 4)
MSVPTQVLGLLLLWLTDARCDIEMTQSPLSLSATPGETVSISCRASEDI
YNALAWYLQKPGRSPRLLIYNTDTLHTGVPDRFSGSGSGTDFTLKISRV
QTEDVGVYFCQHYFHYPRTFGQGTKLELKRSDAQPSVFLFQPSLDELHT
GSASIVCILNDFYPKEVNVKWKVDGVVQNKGIQESTTEQNSKDSTYSLS
STLTMSSTEYQSHEKFSCEVTHKSLASTLVKSFNRSECQRE
felinized anti-NGF antibody (V5_1) VH Sequence:
(SEQ ID NO: 5)
QVQLVESGGDLVQPGGSLRLTCVASGFSLTNNNVNWVRQAPGKGLEWMG
GVWAGGATDYNSAVKSRLTITRDTSKNTVFLQMHSLQSEDTATYYCARD
GGYSSSTLYAMDAWGQGTTVTVSA
felinized anti-NGF antibody (V62) VH Sequence:
(SEQ ID NO: 6)
QVQLVESGGDLVQPGGSLRLTCVASGFSLTNNNVNWVRQAPGKGLEWMG
GVWAGGATDYNSALKSRLTITRDTSKNTVFLQMHSLQSEDTATYYCARD
GGYSSSTLYAMDVWGQGTTVTVSA
felinized anti-NGF antibody (V73) VH Sequence:
(SEQ ID NO: 7)
QVQLVESGGDLVQPGGSLRLTCVASGFSLTNNNVNWVRQAPGKGLEWMG
GVWAGGATDYNSALKSRLTITRDTSKNTVFLQMHSLQSEDTATYYCARD
GGYSSSTLYAMEAWGQGTTVTVSA
felinized anti-NGF antibody (V5_1) HC Sequence:
(SEQ ID NO: 8)
QVQLVESGGDLVQPGGSLRLTCVASGFSLTNNNVNWVRQAPGKGLEWMG
GVWAGGATDYNSAVKSRLTITRDTSKNTVFLQMHSLQSEDTATYYCARD
GGYSSSTLYAMDAWGQGTTVTVSAASTTAPSVFPLAPSCGTTSGATVAL
ACLVLGYFPEPVTVSWNSGALTSGVHTFPAVLQASGLYSLSSMVTVPSS
RWLSDTFTCNVAHPPSNTKVDKTVRKTDHPPGPKPCDCPKCPPPEMLGG
PSIFIFPPKPKDTLSISRTPEVTCLVVDLGPDDSDVQITWFVDNTQVYT
AKTSPREEQFNSTYRVVSVLPILHQDWLKGKEFKCKVNSKSLPSPIERT
ISKAKGQPHEPQVYVLPPAQEELSRNKVSVTCLIKSFHPPDIAVEWEIT
GQPEPENNYRTTPPQLDSDGTYFVYSKLSVDRSHWQRGNTYTCSVSHEA
LHSHHTQKSLTQSPGK

In some embodiments, the monoclonal antibody is a monoclonal antibody that specifically binds EPO, IL-31, VEGF, CD20, Her 2, TNF, IL-2, IgE, IL-33, CD52, CD3, CD19, IL-6, IL-4, IL-4R, IL-13, IL-13R, IL-5, IL-5R, IL-33R, α4β7 integrin, IL-12, IL-23, GMCSF, GMC SFR, PD-1, PD-L1, CTLA-4, B7-1, B7-2, heartworm larva or Gonadotropin-releasing hormone (GnRH).

In some embodiments, the feline anti-NGF antibody is frunevetmab® (Zoetis), the canine anti-NGF antibody is bedinvetmab® (Zoetis), the canine anti-NGF antibody is ranevetmab® (Nexvet), the canine anti-IL-31 antibody and/or felinized form of the anti-IL-31 antibody is lokivetmab® (Zoetis), the canine anti-IL-31 antibody is Lokivetmab® (Zoetis), the canine anti-IL-31 antibody is tirnovetmab® (Kindred), or the canine anti-CD20 antibody is K9LO-133® (Elanco).

In some embodiments, the feline anti-NGF antibody is frunevetmab® (Zoetis).

In some embodiments, the therapeutic protein is GLP-1, insulin, GnRH, MIS/AMH, EPO, IL-4R Trap, IL-5R Trap, ST2R, IL-RA, IL-1RA, or Bone morphogenetic protein (BMP).

In some embodiments, the therapeutic protein is an agonist or antagonist of GLP-1 insulin, GnRH, MIS/AMH, EPO, IL-4R Trap, IL-5R Trap, ST2R, or IL-1RA, IL-31, VEGF, CD20, Her 2, TNF, IL-2, IgE, IL-33, CD52, CD3, CD19, IL-6, IL-4, IL-4R, IL-13, IL-13R, IL-5, IL-5R, IL-33R, α4β7 integrin, IL-12, IL-23, IL-RA, GMCSF, GMCSFR, PD-1, PD-L1, CTLA-4, B7-1, or B7-2.

In an embodiment of the present disclosure, the immune response to a vector comprising a polynucleotide encoding a therapeutic protein may be suppressed by administering the same or similar protein before or slightly after the vector.

In some embodiments, suppression of the immune response is measured by attenuation of anti-transgene product antibody (ATPA) response.

In some embodiments, an ATPA response is attenuated by pre-treatment of a subject with one of two closely related therapeutic proteins.

In some embodiments, an ATPA response is attenuated by pre-treatment of a subject with one of two closely related antibodies.

In some embodiments, an ATPA response is attenuated by administering a therapeutic protein before or slightly after administering a vector comprising a polynucleotide encoding the therapeutic protein.

In some embodiments, an ATPA response is attenuated by administering an immunologically related therapeutic protein before or slightly after administering a vector comprising a polynucleotide encoding the therapeutic protein.

In some embodiments, the therapeutic protein is an antibody.

In some embodiments, an ATPA response is attenuated by administering an antibody before or slightly after administering a vector comprising a polynucleotide encoding the antibody.

In some embodiments, an ATPA response is attenuated by administering an immunologically related antibody before or slightly after administering a vector comprising a polynucleotide encoding the antibody.

In some embodiments, tolerization of the immune response to a vector comprising a polynucleotide encoding a therapeutic protein is attenuated by administering an immunologically related therapeutic protein before or slightly after administering the vector.

In some embodiments, tolerization of the immune response to a vector comprising a polynucleotide encoding an antibody is attenuated by administering an immunologically related antibody before or slightly after administering the vector.

In some embodiments, the immunologically related antibody shares at least 85%, at least 90%, at least 91%, at least 92%, at least 93%, at least 94%, at least 95%, at least 96%, at least 97%, at least 98%, at least 99%, or 100% homology to the antibody encoded by a polynucleotide and administered in a vector.

In some embodiments, the therapeutic protein administered before the vector is frunevetmab® or an immunologically related variant thereof.

In some embodiments, the therapeutic protein administered before the vector is an anti-NGF antibody or an immunologically related variant thereof.

In some embodiments, a tolerizing dose of therapeutic protein is administered to the subject before or slightly after administration of a therapeutic protein encoded by a polynucleotide and administered in a vector.

In some embodiments, a tolerizing dose of antibody is administered to the subject before or slightly after administration of a therapeutic protein encoded by a polynucleotide and administered in a vector.

In some embodiments, the therapeutic protein administered to the subject as a tolerizing dose is a related therapeutic protein with highly similar, but not identical, nucleotide sequence to the therapeutic protein administered in a vector.

In some embodiments, the related therapeutic protein shares at least 85%, at least 90%, at least 91%, at least 92%, at least 93%, at least 94%, at least 95%, at least 96%, at least 97%, at least 98%, at least 99%, or 100% homology to the therapeutic protein encoded by a polynucleotide and administered in a vector.

In some embodiments, the antibody administered to the subject as a tolerizing dose is a related antibody with highly similar, but not identical, nucleotide sequence to the antibody administered in a vector.

In some embodiments, the related antibody shares at least 85%, at least 90%, at least 91%, at least 92%, at least 93%, at least 94%, at least 95%, at least 96%, at least 97%, at least 98%, at least 99%, or 100% homology to the antibody encoded by a polynucleotide and administered in a vector. In some embodiments, the therapeutic protein is a fusion protein. A fusion protein is a protein consisting of at least two domains that are encoded by separate genes that have been joined so that they are transcribed and translated as a single unit, producing a single polypeptide (e.g., Fc-fusion proteins, albumin-fusion proteins, and other fusion protein known in the art).

In some embodiments, the therapeutic protein is administered intramuscularly.

In some embodiments, the therapeutic protein is administered intravenously.

In some embodiments, the therapeutic protein is administered subcutaneously.

In some embodiments, the therapeutic protein or a functional fragment of the therapeutic protein shares at least 70%, at least 75%, at least 80%, at least 85%, at least 90%, at least 95%, at least 99%, or 100% sequence identity to an endogenous protein of the subject or a functional fragment of the endogenous protein.

In some embodiments, the therapeutic protein or a functional fragment of the therapeutic protein shares at least 95% sequence identity to an endogenous protein of the subject or a functional fragment of the endogenous protein.

In some embodiments, the subject is a feline.

In some embodiments, the subject is a canine.

In some embodiments, the subject is a human.

In some embodiments, the therapeutic protein is a feline monoclonal antibody or a felinized monoclonal antibody.

In some embodiments, the therapeutic protein is a canine monoclonal antibody or a caninized monoclonal antibody.

In some embodiments, the subject is a juvenile or an adult.

In some embodiments, the subject is not a newborn.

In some embodiments, the therapeutic protein is administered at a dose of less than about 50 mg/kg, less than about 25 mg/kg, less than about 10 mg/kg, less than about 5 mg/kg, less than about 1 mg/kg, or less than about 0.5 mg/kg.

In some embodiments, the therapeutic protein is administered at a dose of at least about 0.01 mg/kg, at least about 0.05 mg/kg, at least about 0.1 mg/kg, at least about 1 mg/kg, at least about 2 mg/kg, at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, or at least about 50 mg/kg.

In some embodiments, the therapeutic protein is administered at a dose of about 0.1 mg/kg to about 50 mg/kg, about 1 mg/kg to about 50 mg/kg, about 0.1 mg/kg to about 25 mg/kg, about 1 mg/kg to about 25 mg/kg, about 1 mg/kg to about 10 mg/kg, or about 5 mg/kg to about 10 mg/kg.

In some embodiments, the therapeutic protein is administered at a dose of about 0.01 mg/kg, about 0.05 mg/kg, about 0.1 mg/kg, about 1 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg.

In some embodiments, the therapeutic protein is administered at a sub-therapeutically effective dose for the therapeutic protein.

In some embodiments, the therapeutic protein is administered to the subject for less than 6 weeks, less than 5 weeks, less than 4 weeks, less than 3 weeks, less than 2 weeks, less than 1 week, less than 5 days, less than 3 days, or less than one day before the vector is administered.

In some embodiments, the subject is administered at most one dose, at most two doses, at most three doses, at most four doses, or at most five doses of therapeutic protein before the vector is administered.

In some embodiments, the therapeutic protein composition is administered to the subject at a dose effective to suppress an immune response to the therapeutic protein encoded by the vector.

In some embodiments, the immune response is an antibody-based immune response of the subject to the therapeutic protein in response to expression of the therapeutic protein by cells transduced with the vector in the subject.

In some embodiments, the method reduces the host antibody response by at least about 10%, at least about 20%, at least about 30%, at least about 40%, at least about 50%, at least about 60%, at least about 70%, at least about 75%, at least about 80%, at least about 90%, at least about 95%, or 100%.

In some embodiments, the subject is treatment-naïve to the therapeutic protein. In some embodiments, the subject is treatment-naïve to the vector. In some embodiments, the subject is treatment-naïve to the therapeutic protein and to the vector.

In some embodiments, the therapeutic protein is recognized by the immune system as a self-antigen.

In some embodiments, the therapeutic protein is administered at a time which would allow measurable levels of the therapeutic protein to be present at the time of administration of the vector.

In some embodiments, the therapeutic protein is administered before expression of the therapeutic protein by the vector would be expected.

As noted elsewhere herein, the present invention is predicated, at least in part, on the inventors' surprising finding that treatment of subjects with a purified recombinant version of the therapeutic protein is effective in preventing anti-transgene product antibody formation following subsequent intramuscular administration of the vector.

The anti-transgene product antibody (ATPA) response to a vector-delivered protein can be suppressed by pre-treatment and/or concurrent treatment of the subject with a purified recombinant form of the same protein or functional variant of the therapeutic protein, optionally an immunologically related variant thereof.

The subject may be administered the recombinant therapeutic protein subcutaneously and subsequently administered the vector intramuscularly.

The subject may be administered the recombinant therapeutic protein intramuscularly and subsequently administered the vector subcutaneously.

The subject may be administered the recombinant therapeutic protein subcutaneously and subsequently administered the vector subcutaneously.

The subject may be administered the recombinant therapeutic protein intramuscularly and subsequently administered the vector intramuscularly.

### Antigen-Binding Molecule

Disclosed herein is an antigen-binding molecule that is capable of binding specifically to a therapeutic protein, wherein the antigen-binding molecule comprises an immunoglobulin heavy chain variable domain (VH) and an immunoglobulin light chain variable domain (VL).

By “antigen-binding molecule” is meant a molecule that has binding affinity for a target antigen. It will be understood that this term extends to immunoglobulins, immunoglobulin fragments and non-immunoglobulin-derived protein frameworks that exhibit antigen-binding activity. Illustrative examples of suitable antigen-binding molecules include antibodies and antigen-binding fragments thereof. Preferably, the antigen-binding molecule binds specifically to a protein so as to neutralize, or substantially neutralize, its activity. The term “neutralize” is understood to mean that the antigen-binding molecule will bind to the protein and inhibit, reduce, abrogate, block or otherwise prevent the ability of the protein to bind to its native receptor.

In an embodiment, the antigen-binding molecule, as described herein, is conjugated to another molecule or moiety, including functional moieties (e.g., toxins), detectable moieties (e.g., fluorescent molecules, radioisotopes), small molecule drugs and polypeptides.

The term “antibody”, as used herein, is understood to mean any antigen-binding molecule or molecular complex comprising at least one complementarity determining region (CDR) that binds specifically to, or interacts specifically with, the target antigen. The term “antibody” includes full-length immunoglobulin molecules comprising two heavy (H) chains and two light (L) chains inter-connected by disulfide bonds, as well as multimers thereof (e.g., IgM). Each heavy chain comprises a heavy chain variable region (which may be abbreviated as HCVR, VH or VH) and a heavy chain constant region. The heavy chain constant region typically comprises three domains—CH1, CH2 and CH3. Each light chain comprises a light chain variable region (which may be abbreviated as LCVR, VL, VK, VK or VL) and a light chain constant region. The light chain constant region will typically comprise one domain (CL1). The VH and VL regions can be further subdivided into regions of hypervariability, termed complementarity determining regions (CDRs), interspersed with regions that are more conserved, also referred to as framework regions (FR).

Suitable antibodies include antibodies of any class, such as IgG, IgA, or IgM (including sub-classes thereof). There are five major classes of immunoglobulins: IgA, IgD, IgE, IgG, and IgM, characterized by heavy-chain constant regions α, β, ε, γ, and μ, respectively. Several antibody classes may be further divided into subclasses (isotypes), e.g., IgG1, IgG2, IgG3, IgG4, IgA1 and IgA2. The subunit structures and three-dimensional configurations of different classes of immunoglobulins will be well known to persons skilled in the art.

As used herein, the term “complementarity determining region” (CDR) refers to the region of an immunoglobulin variable domain that recognizes and binds to the target antigen. Each variable domain may comprise up to three CDR sequences, identified as CDR1, CDR2 and CDR3.

The term “therapeutic protein” in the context of the present invention, means a recombinant protein (e.g., an immunoglobulin, protein fragment, engineered protein, or enzyme) that has been sufficiently purified or isolated from contaminating proteins, lipids, and nucleic acids present in a liquid culture medium or from a host cell (e.g., from a mammalian, yeast, or bacterial host cell) and biological contaminants (e.g., viral and bacterial contaminants), and can be formulated into a pharmaceutical agent without any further substantial purification and/or decontamination step. The therapeutic protein is homologous to an endogenous protein of the subject and does not generate an immune response in the intended recipient, i.e., the therapeutic protein is recognized by the immune system as a self-antigen.

The terms “functional variant”, “functional fragment”, and “functional fragment of a variant” (also termed “functional variant fragment”) in the context of the present invention, mean that the fragment of the protein, the variant of the protein, or the fragment of a variant of the protein fulfils at least one, preferably more than one, function of the endogenous host protein of which the variant, the fragment, or the fragment of a variant is derived. Further, the functional variant may have the same or substantially the same biochemical or physiological effect in the subject as the endogenous host protein, or may have the same or substantially similar molecular mechanisms as the endogenous host protein. The functional variant may share at least 80% identity, at least 90% identity, at least 95% identity, or 100% sequence identity to the endogenous host protein.

The term “homolog” refers to the biological homology between protein sequences, defined in terms of shared ancestry in the evolutionary history of life. This homology may be inferred from amino acid similarity as significant sequence similarity is strong evidence that the sequences are from a common ancestral sequence. A protein homolog may also comprise a variation in its amino acid sequence while maintaining at least one function of the endogenous host protein.

The term “immunologically related” refers to a protein that shares the same dominant B and T cell epitopes as the endogenous host protein.

The terms “antigen-binding fragment”, “antigen-binding portion”, “antigen binding domain”, “antigen-binding site” and the like are used interchangeably herein to refer to a part of an antigen-binding molecule that retains the ability to bind to the target antigen. These terms include naturally occurring, enzymatically obtainable, synthetic or genetically engineered (recombinant) polypeptides and glycoproteins that specifically bind to the therapeutic protein to form a complex.

The term “anti-transgene product antibody” (ATPA) response refers to a humoral immune response to a transgene which has been delivered to the subject via vector gene transfer. The ATPA response may be an antibody-based immune response of the subject to the therapeutic protein in response to expression of the therapeutic protein by cells transduced with the vector in the subject.

Non-limiting examples of suitable antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated CDR such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g. monovalent nanobodies, bivalent nanobodies, etc.), and small modular immunopharmaceuticals (SMIPs), are also encompassed by the terms “antibody fragment” or “antigen-binding fragment,” as used herein.

In some embodiments, the antigen-binding molecule or antigen-binding fragment thereof is modified for compatibility with the target species. Thus, in an embodiment, the antigen-binding molecule or antigen-binding fragment thereof is humanized, caninized, felinized or equinized.

In a particular embodiment, a NGF antibody or antigen-binding fragment thereof is humanized, caninized, felinized or equinized.

By “humanized” is meant that the antigen-binding molecule comprises an amino acid sequence that is compatible with humans, such that the amino acid sequence is unlikely to be seen as foreign by the immune system of a human subject. By “caninized” is meant that the antigen-binding molecule comprises an amino acid sequence that is compatible with canine, such that the amino acid sequence is unlikely to be seen as foreign by the immune system of a canine subject. By “felinized” is meant that the antigen-binding molecule comprises an amino acid sequence that is compatible with feline, such that the amino acid sequence is unlikely to be seen as foreign by the immune system of a feline subject. By “equinized” is meant that the antigen-binding molecule comprises an amino acid sequence that is compatible with equine, such that the amino acid sequence is unlikely to be seen as foreign by the immune system of an equine subject.

Suitable methods of designing and producing recombinant antibodies or antigen binding molecules that are compatible with the target species will be familiar to persons skilled in the art, illustrative examples of which are described in WO 2006/131951, WO 2012/153122, WO 2013/034900, WO 2012/153121 and WO 2012/153123, the contents of which are incorporated herein by reference in their entirety.

The phrase “specifically binds” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antigen-binding molecule binds to a particular antigenic determinant, thereby identifying its presence. Specific binding to an antigenic determinant under such conditions requires an antigen-binding molecule that is selected for its specificity to that determinant. This selection may be achieved by subtracting out antigen-binding molecules that cross-react with other molecules. A variety of immunoassay formats may be used to select antigen-binding molecules (e.g., immunoglobulins) [such that they are specifically immunoreactive with a particular antigen. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are also well known in the art (see, for example, Harlow and Lane, supra); Friefelder, “Physical Biochemistry: Applications to biochemistry and molecular biology” (W. H. Freeman and Co. 1976).

The terms “polypeptide”, “peptide”, or “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide.

Also disclosed herein is a chimeric molecule comprising a therapeutic protein, as herein described, and a heterologous moiety. In some embodiments, the heterologous moiety may be a detectable moiety, a half-life extending moiety, or a therapeutic moiety. Thus, as used herein, a “chimeric” molecule is one which comprises one or more unrelated types of components or contains two or more chemically distinct regions which can be conjugated to each other, fused, linked, translated, attached via a linker, chemically synthesized, expressed from a nucleic acid sequence, etc. For example, a peptide and a nucleic acid sequence, a peptide and a detectable label, unrelated peptide sequences, and the like. In embodiments in which the chimeric molecule comprises amino acid sequences of different origin, the chimeric molecule includes (1) polypeptide sequences that are not found together in nature (i.e., at least one of the amino acid sequences is heterologous with respect to at least one of its other amino acid sequences), or (2) amino acid sequences that are not naturally adjoined.

The term “polynucleotide” or “nucleic acid” are used interchangeably herein to refer to a polymer of nucleotides, which can be mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

The terms “host”, “host cell”, “host cell line” and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the antigen binding molecules of the present invention. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue. In one embodiment, the host cell is a CHO or HEK293 cell line.

Methods for producing a modified therapeutic protein-binding molecule, as described herein, is provided, such methods comprising culturing the host cell disclosed herein and recovering the antigen-binding molecule from the host cell or culture medium. In a particular embodiment, the modified therapeutic protein-binding molecule is a NGF-binding molecule.

### Vectors

As used herein, the terms “vector” and “construct”, which are used interchangeably, may be nucleic acid molecules, preferably DNA molecules derived, for example, from a plasmid, bacteriophage, or virus, into which a nucleic acid sequence may be inserted or cloned. A vector may contain one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or two or more vectors, which together contain the total DNA to be introduced into the genome of the host cell, or a transposon. The choice of the vector will typically depend on the compatibility of the vector with the host cell into which the vector is to be introduced.

In some embodiments, the vector may be a naked polynucleotide (e.g., for use in electroporation or microneedle administration), a liposome of lipid-based particle, or a virus (e.g., lentivirus, AAV, adenovirus, or other viral vector known in the art).

In some embodiments, the vector is a recombinant adeno-associated virus (AAV) vector that enables persistent expression of the recombinant therapeutic protein in the subject.

Adeno-associated viral vectors carrying therapeutic protein expression constructs have been developed for use in feline, canine, human, and equine subjects. Viral vector therapeutics have the advantage of convenience, insofar as only a single administration or a lower number of doses may be required to treat the subject, as opposed to requiring recurrent administration of recombinant therapeutic protein, thereby further improving quality of life. The therapeutic protein constructs described herein suitably provide a sequence that is native to the subject to be treated, as this will typically reduce or otherwise avoid the risk of the subject developing an immune response to a non-native protein.

Also disclosed herein is an expression construct comprising a nucleic acid sequence comprising the therapeutic protein, as described herein, operably linked to one or more regulatory sequences.

By “control element”, “control sequence”, “regulatory sequence” and the like, as used herein, mean a nucleic acid sequence (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

Liver-specific promoters which may be used include, but are not limited to alpha 1 anti-trypsin (A1AT) (see, e.g., The Liver Specific Gene Promoter Database, Cold Spring Harbor, http://rulai.schl.edu/LSPD), human albumin (humAlb) (Miyatake et al., J. Virol., 71:512432 (1997)), and hepatitis B virus core promoter (Sandig et al., Gene Ther., 3:10029 (1996)). TTR minimal enhancer/promoter, alpha-antitrypsin promoter, LSP (845 nt) 25 (requires intron-less scAAV). In one embodiment, the liver-specific promoter thyroxin binding globulin (TBG) is used. Other promoters, such as viral promoters, constitutive promoters, regulatable promoters (see, e.g., WO 2011/126808 and WO 2013/04943), or a promoter responsive to physiologic cues may be used may be utilized in the vectors described herein.

In some embodiments, the feline anti-NGF monoclonal antibody AAV delivered transgene is under the control of a liver-specific promoter (TBG).

In some embodiments, the feline anti-NGF monoclonal antibody AAV delivered transgene is under the control of a non-tissue specific promoter (CB7).

Also disclosed herein is an expression construct comprising a nucleic acid sequence encoding a NGF-binding molecule, as described herein, operably linked to one or more regulatory sequences.

As used herein, an “expression construct” refers to a nucleic acid molecule which comprises coding sequences for the therapeutic protein, promoter, and may include other regulatory sequences therefor, which cassette may be engineered into a genetic element and/or packaged into the capsid of a viral vector (e.g., a viral particle). Typically, such an expression cassette for generating a viral vector contains the therapeutic protein construct sequences described herein flanked by packaging signals of the viral genome and other expression control sequences such as those described herein. Any of the expression control sequences can be optimized for a specific species using techniques known in the art including, e.g., codon optimization, as described elsewhere herein.

In an embodiment, the expression control sequence comprise a tissue-specific promoter. Suitable tissue-specific promoters will be familiar to persons skilled in the art. In an embodiment, the tissue-specific promoter is a kidney-specific promoter or a muscle-specific promoter. In an embodiment, the tissue-specific promoter is selected from a Nkcc2 promoter, uromodulin promoter, Ksp-cadherin promoter and THP gene promoter.

In an embodiment, the AAV capsid further comprises one or more of an intron, a Kozak sequence, a polyA, and post-transcriptional regulatory elements.

Suitable AAV capsids will be familiar to persons skilled in the art, illustrative examples of which include AAV1, AAV5, AAV6, AAV8, AAVrh64R1, AAV9, AAVrh91, AAVhu.37, AAV3b, AAV3b.AR2.12 and AAVrh10. In some embodiments, the AAV capsid is an AAV1, AAV5, AAV6, AAV8, AAVrh64R1, AAV9, AAVrh91, AAVhu.37, AAV3b, AAV3b.AR2.12 or AAVrh10.

In some embodiments, the AAV capsid is an AAV1 capsid.

In some embodiments, the AAV capsid is an AAVrh91 capsid.

An adeno-associated virus (AAV) viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. AAV serotypes may be selected as sources for capsids of AAV viral vectors (DNase resistant viral particles) including, e.g., AAV1, AAV2, AAV3, AAV4, AAV5, AAV6, AAV6.2, AAV7, AAV8, AAV9, rh10, AAVrh64R1, AAVrh64R2, rh8, rh.10, variants of any of the known or mentioned AAVs or AAVs yet to be discovered. In one embodiment, the AAV is an AAV8 capsid, or a variant thereof. See, e.g., US 2007/0036760 A1; US 2009/0197338 A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10). Alternatively, a recombinant AAV based upon any of the recited AAVs, may be used as a source for the AAV capsid. These documents also describe other AAV which may be selected for generating AAV and are incorporated by reference. In some embodiments, an AAV cap for use in the viral vector can be generated by mutagenesis (i.e., by insertions, deletions, or substitutions) of one of the aforementioned AAV Caps or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned Caps. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3). In one embodiment, the AAV capsid shares at least 95% identity with the AAV8 vp3. In another embodiment, a self-complementary AAV is used.

Methods for generating and isolating AAV viral vectors suitable for delivery to a subject are known in the art (see, e.g., U.S. Pat. Nos. 7,790,449; 7,282,199; WO 2003/042397; WO 2005/033321, WO 2006/110689; U.S. Pat. No. 7,588,772 and WO 2017/040524, the contents of which are incorporated herein by reference in their entirety). In an embodiment, a producer cell line is transiently transfected with a construct that encodes the transgene flanked by inverted terminal repeats (ITR) and a construct(s) that encodes rep and cap. In another embodiment, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al. (2009, Human Gene Therapy 20:922-929, the entire contents of which are incorporated herein by reference). Methods of making and using these and other AAV production systems are also described in the following U.S. patents, the entire contents of which are incorporated herein by reference: U.S. Pat. Nos. 5,139,941; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, Adv. Biochem. Engin/Biotechnol. 99: 119-145; Buning et al., 2008, J. Gene Med.10:717-733). Suitable methods for constructing any of the embodiments described herein will be familiar to persons skilled in the art of nucleic acid manipulation, illustrative examples of which include genetic engineering, recombinant engineering, and synthetic techniques (see, e.g., Green and Sambrook et al, Molecular Cloning: A Laboratory Manual, Cold Spring Harbor Press, Cold Spring Harbor, NY (2012)). Similarly, methods of generating rAAV virions will be well known to persons skilled in the art (see, e.g., K. Fisher et al, (1993) J. Virol., 70:520-532 and U.S. Pat. No. 5,478,745).

The viral vectors described herein may be delivered in a single composition or multiple compositions. Optionally, two or more different AAV may be delivered, or multiple viruses (see, e.g., WO 2011/126808 and WO 2013/049493). In another embodiment, multiple viruses may contain different replication-defective viruses (e.g., AAV and adenovirus).

### Modes of Administration and Dosing

The disclosed viral particles and therapeutic proteins may be administered in a number of ways depending upon whether local or systemic treatment is desired.

In general, administration may be topical, parenteral, or enteral. The compositions of the disclosure are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intranasal, intratracheal, intraarterial, intrathecal, intraventricular, intraurethral, intracranial, intratumoral, intraocular, intradermal, intrasynovial injection or infusions; and kidney dialytic infusion techniques. In an embodiment, the compositions of the present disclosure comprise intravenous administration. In an embodiment, the compositions of the present disclosure comprise intramuscular administration. In an embodiment, the compositions of the present disclosure comprise subcutaneous administration.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The replication-defective viruses can be formulated with a physiologically acceptable carrier for use in gene transfer and gene therapy applications. In the case of AAV viral vectors, quantification of the genome copies (“GC”) may be used as the measure of the dose contained in the formulation or suspension. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually poly A signal).

In some embodiments, the amount of viral genome may be delivered in split doses. In an embodiment, the constructs may be delivered in volumes from 1 μL to about 100 mL for a veterinary subject (see e.g., Diehl et al, 2001, J. Applied Toxicology, 21:15-23 for a discussion of good practices for administration of substances to various veterinary animals, the contents of which are incorporated herein by reference in their entirety). Thus, as used herein, the term “dosage” or “dose” can refer to the total dosage delivered to the subject in the course of treatment, or the amount delivered in a single (of multiple) administration.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. An antigen-binding molecule of the present disclosure can be administered on multiple occasions. Intervals between single dosages can be daily, weekly, monthly or yearly. Intervals can also be irregular as indicated by measuring blood levels of modified polypeptide or antigen in the patient. Alternatively, the antigen-binding molecule can be administered as a sustained release formulation, in which case less frequent administration is required. Dosage and frequency vary depending on the half-life of the polypeptide in the patient.

In certain embodiments, the therapeutic protein is administered by injection (e.g., subcutaneously, intravenously, or intramuscularly) at a dose of about 0.01 to 50 mg/kg, about 0.01 to 0.1 mg/kg, e.g., about 0.1 to 1 mg/kg, about 1 to 5 mg/kg, about 5 to 25 mg/kg, about 10 to 40 mg/kg, or about 15 to 50 mg/kg. In an embodiment, the therapeutic protein is administered at a dose of about 2 mg/ml. The dosing schedule can vary from e.g., once a week to once every 2, 3, or 4 weeks.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### Pharmaceutical Compositions

Also disclosed herein is a pharmaceutical composition comprising the therapeutic protein and/or a vector, as described herein, and a pharmaceutically acceptable carrier.

Suitable methods fort delivering the recombinant vectors to host cells will be known to persons skilled in the art. The vector, preferably suspended in a physiologically compatible carrier, diluent, excipient and/or adjuvant, may be administered to a feline, canine, human, or equine subject. Suitable carriers may be readily selected by persons skilled in the art in view of the indication for which the transfer virus is directed. For example, a suitable carrier includes saline, which may be formulated with a variety of buffering solutions (e.g., phosphate buffered saline). Other illustrative examples of suitable carriers include sterile saline, lactose, sucrose, calcium phosphate, gelatin, dextran, agar, pectin, peanut oil, sesame oil, and water.

The compositions described herein may contain, in addition to the vector, other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In the context of administering viral particles, the amount of viral particles and time of administration of such particles will be within the purview of the skilled artisan having benefit of the present teachings. In some embodiments, the administration of therapeutically-effective amounts of the disclosed compositions may be achieved by a single administration, such as for example, a single injection of sufficient numbers of viral particles to provide therapeutic benefit to the patient undergoing such treatment. In some embodiments, the subject is provided multiple, or successive administrations of the viral vector composition, either over a relatively short, or a relatively prolonged period of time, as may be determined by the medical practitioner overseeing the administration of such compositions. In some embodiments, a subject may be administered two or more different viral vector compositions, either alone, or in combination with one or more other therapeutic drugs to achieve the desired effects of a particular therapy regimen. The phrase “in combination” may comprise at the same time or at different times within a short period of time, e.g., within one week, one day, twelve hours, six hours, one hour, thirty minutes, ten minutes, five minutes, or one minute.

In the context of administering therapeutic proteins, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin and/or by the maintenance of the required particle size. In specific embodiments, an agent of the present disclosure may be conjugated to a vehicle for cellular delivery. In these embodiments, the agent may be encapsulated in a suitable vehicle to either aid in the delivery of the agent to target cells, to increase the stability of the agent, or to minimize potential toxicity of the agent. As will be appreciated by a skilled artisan, a variety of vehicles are suitable for delivering an agent of the present disclosure. Non-limiting examples of suitable structured fluid delivery systems may include nanoparticles, liposomes, microemulsions, micelles, dendrimers and other phospholipid-containing systems. Methods of incorporating agents of the present disclosure into delivery vehicles are known in the art. Although various embodiments are presented below, it will be appreciated that other methods known in the art to incorporate an antigen-binding molecule, as described herein, into a delivery vehicle are contemplated.

The present compositions of recombinant therapeutic protein and/or viral particles may be administered in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the disease is degenerative joint disease (DJD). In some embodiments, the disease is osteoarthritis (OA).

The pharmaceutical compositions of the invention may include an effective amount of a therapeutic protein (e.g., a NGF-binding molecule) as disclosed herein. A “therapeutically effective amount” refers to an amount effective, at dosages and for periods of time necessary, to achieve the desired therapeutic result. A therapeutically effective amount of the agent may vary according to factors such as the disease state, age, sex, and weight of the individual, and the ability of the agent to elicit a desired response in the individual. A therapeutically effective amount is also one in which any toxic or detrimental effects of the agent is outweighed by the therapeutically beneficial effects. Alternatively, this property of a composition can be evaluated by examining the ability of the compound to inhibit, for example in in vitro by assays known to the skilled practitioner.

In an embodiment, the therapeutic protein is administered to the subject at a dose effective to suppress a host antibody immune response to the therapeutic protein encoded by the vector.

In some embodiments, the immune response is an antibody-based immune response of the subject to the therapeutic protein in response to expression of the therapeutic protein by cells transduced with the vector in the subject.

In some embodiments, the method reduces the host antibody response by at least about 20%, at least about 30%, at least about 40%, at least about 50%, at least about 60%, at least about 70%, at least about 75%, at least about 85%, at least about 90%, at least about 95%, or at least about 99%.

The terms “subject”, “patient”, “host” or “individual” used interchangeably herein, refer to any subject, including a vertebrate subject and a mammalian subject, for whom therapy is desired. Suitable vertebrate animals that fall within the scope of the invention include, but are not restricted to, any member of the subphylum Chordata including primates (e.g., humans, monkeys and apes, and includes species of monkeys such as from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In one embodiment, the subject is a human subject. In another embodiment, the subject is a canine subject. In another embodiment, the subject is a feline subject. In another embodiment, the subject is an equine subject.

### ****

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described.

While illustrative embodiments have been illustrated and described, it will be appreciated that various changes can be made therein without departing from the spirit and scope of the invention.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what is regarded as the invention.

### Example 1: Assessment of Anti-Transgene Product Antibodies (ATPA) in Cat Serum

This example demonstrates that IM administration of an AAV containing a feline anti-NGF mAb transgene at three different doses results in a high incidence of ATPA responses that affect circulating levels of the antibody. Three cohorts of cats (n=4/group) were given a single IM dose of an AAV containing a feNGF mAb transgene under the control of a CB7 promoter at either 1×10¹¹ gc/kg, 5×10¹¹ gc/kg or 3×10¹² gc/kg. Serum levels of the feNGF mAb and ATPA response were monitored for up to 70 days using methods as described herein.

### Quantification of Feline Anti-NGF Antibody in Cat Serum

The concentration of FeNGF mAb in cat serum was determined using an NGF-binding ELISA. ELISA plates were coated with 0.1 μg/ml muNGF and blocked with PBS/0.05% Tween 20/1% BSA. muNGF coated wells were incubated for 1 h at room temperature with serum diluted in PBS/0.05% Tween 20/1% BSA in the amount of 100 μl/well. FeNGF antibody concentrations ranging from 1.56 ng/ml to 100 ng/ml were used to establish a binding curve. Well plates were washed following incubation and were then incubated with a 1/10,000 dilution of goat anti-feline IgG-HRP in PBS/0.05% Tween 20/1% BSA. Plates were washed with PBS/0.05% Tween 20 and developed by the addition of 3,3′,5,5′-tetramethylbenzidine (TMB) substrate. Development was stopped by the addition of 2M H2SO4 and the absorbance read at 450 nm and background was subtracted from the resulting reading.

A bridging Immunoassay with acid dissociation was used to evaluate the presence of anti-(anti-feNGF mAb) antibodies in the cat serum. Samples were pre-treated with acid to disrupt any existing ATPA-feNGF mAb complexes before assessment in the bridging assay. Samples were diluted to 2.5% in 300 mM acetic acid to enable ATPA-feNGF mAb complex dissociation before analysis. Acidified samples were incubated for 40 min with shaking at ambient temperature. 25 μl of the acidified samples were transferred to wells of a 96 well plate containing 90 μL of master-mix reagent (0.125 μg/mL of biotinylated feNGF mAb and 0.125 μg/mL of ruthenylated feNGF mAb with 1% Meso Scale Discovery (MSD) Blocker A in PBS-T) and 11 μL 1.2M Tris solution (pH 9.5). Acidified samples plus master-mix reagents were incubated at ambient temperature in the dark for 60 min with shaking. Simultaneously, Streptavidin-coated MSD plates were blocked for 60 min at ambient temperature with 200 μL/well of PBS-T buffer containing 3% (MSD) Blocker A. The Streptavidin-coated MSD plates were then washed and 25 μl of the acidified sample plus master-mix reagent were transferred to the plates which were then incubated at ambient temperature in the dark for 60-90 min with shaking. The MSD plates were washed and then 150 μL of 2×MSD Read T-Buffer was added per well before the plates were read on an MSD MESO QuickPlex SQ 120 instrument. The resulting response was recorded as Relative Light Units (RLU).

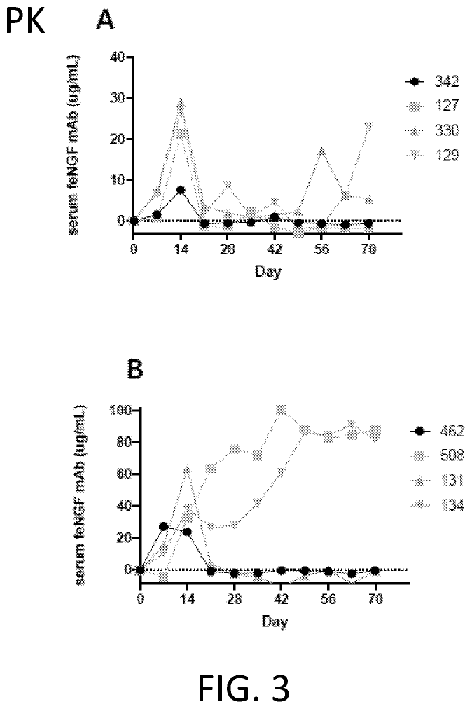

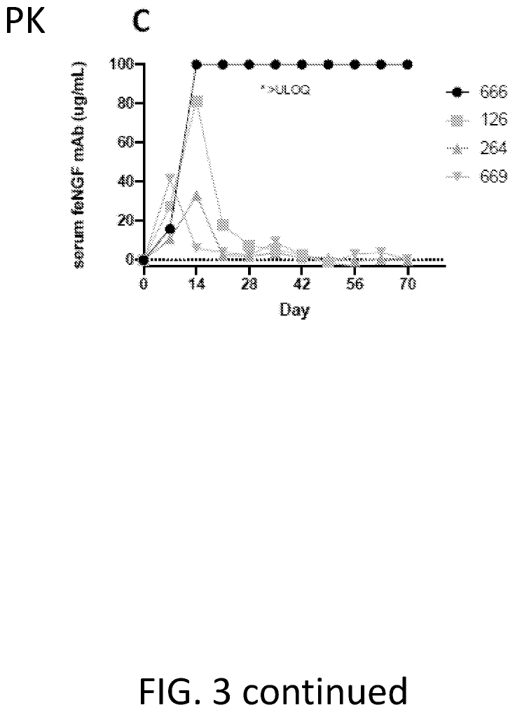

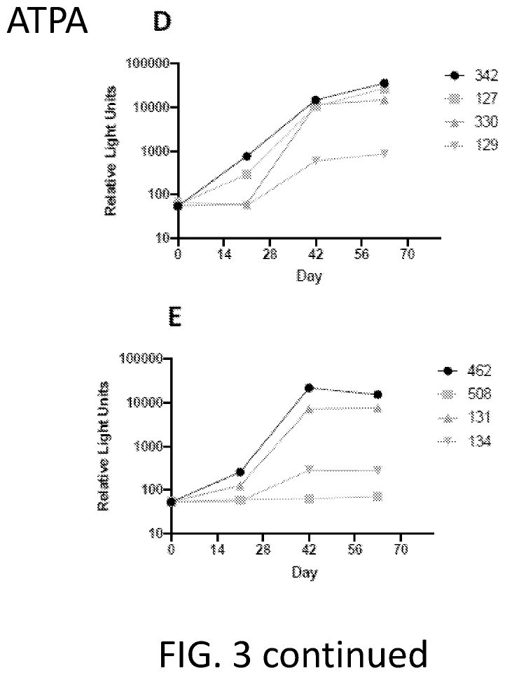

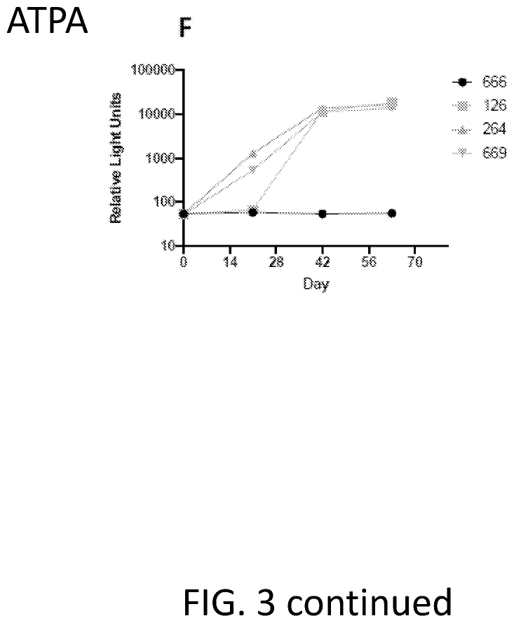

As shown in FIGS. 3A-3F, cats were administered AAV containing the feNGF mAb transgene under the control of a CB7 promoter IM at a dose of 1×10¹¹ gc/kg (FIG. 3A, FIG. 3D), 5×10¹¹ gc/kg (FIG. 3B, FIG. 3E), or 3×10¹² gc/kg (FIG. 3C, FIG. 3F) at day 0. Serum levels of feNGF mAb were determined using an NGF-binding ELISA as described above. ATPA were detected using a bridging immunoassay. The data suggest that the majority of animals generated a strong ATPA response following AAV administration. The ATPA response often correlated with a loss of detection of the feNGF mAb in the serum.

### Example 2: Pre-Treatment Prior to Administration of AAV Containing the feNGF mAb

This example demonstrates that pre-treatment with a recombinant protein of interest can abrogate the immune response directed to that protein when it is delivered by AAV.

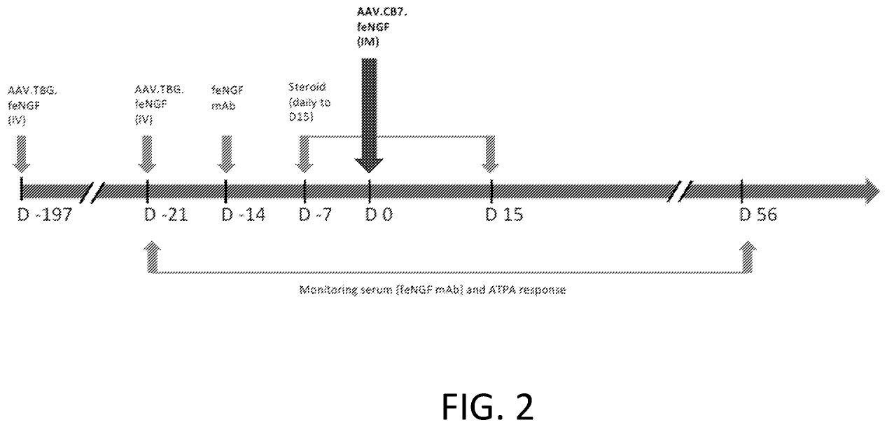

As shown in FIG. 2, cats were administered pre-treatments at various times prior to the administration of AAV containing the feNGF mAb transgene. These pre-treatments included:
Intravenous administration of AAV containing the feNGF mAb transgene under the control of a liver-specific promoter (TBG). AAV.TBG.feNGF was given at a dose of 1×10¹² gc/kg at day −197
Intravenous administration of AAV containing the feNGF mAb transgene under the control of a liver-specific promoter (TBG). AAV.TBG.feNGF was given at a dose of 5×10¹¹ gc/kg at day −21
Subcutaneous administration of recombinant purified feNGF mAb at a dose of 2 mg/kg at day −14 Daily oral administration of prednisolone at 1 mg/kg from days −7 to day 9. Prednisolone dose was tapered off to 0 mg/kg from day 10 to day 16.

At day 0, cats were intramuscularly administered AAV containing the feNGF mAb transgene under the control of a CB7 promoter (not tissue specific) at a dose of 2.5×10¹¹ gc/kg. Serum levels of feNGF mAb were determined using an NGF-binding ELISA and ATPA were detected using a bridging immunoassay as described above.

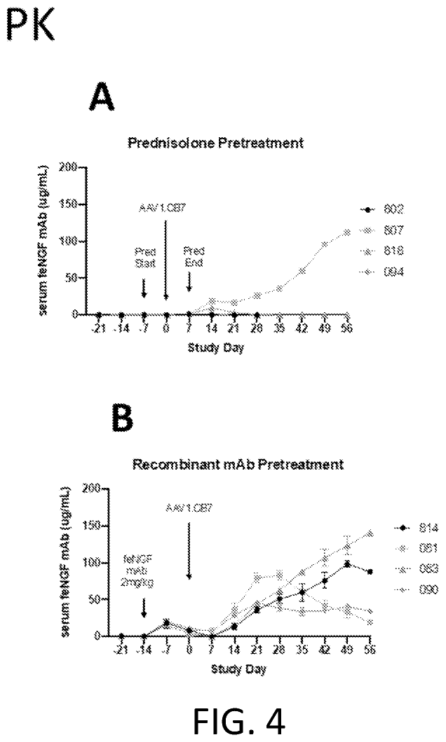

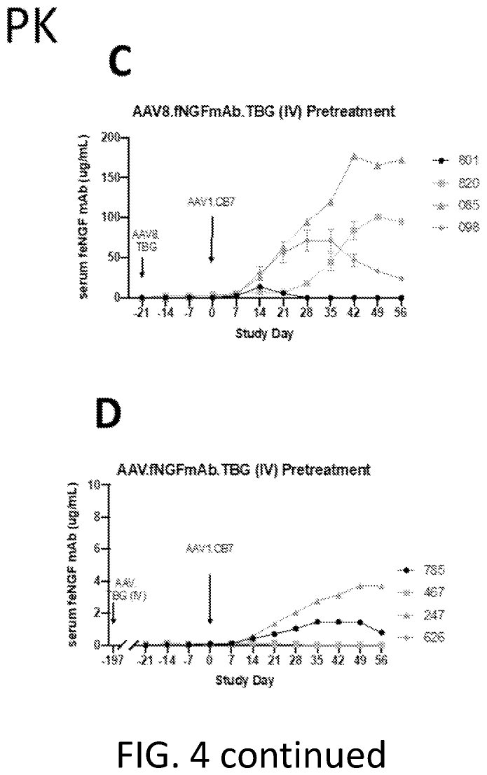

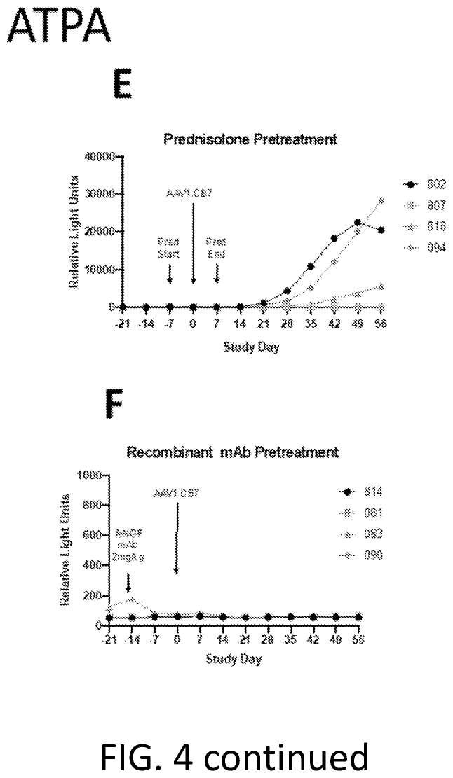

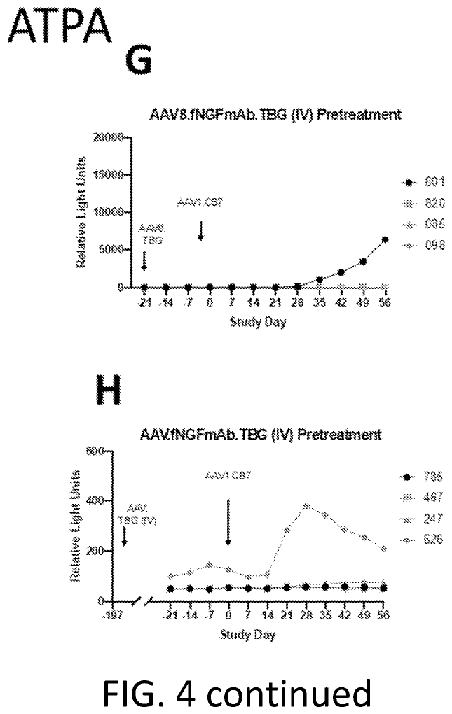

As shown in FIGS. 4A-4H, cats were administered pre-treatments as listed above in FIG. 2. AAV containing the feNGF mAb transgene under the control of a CB7 promoter was administered intramuscularly at day 0 at a dose of 2.5×10¹¹ gc/kg. Serum levels of feNGF mAb were determined using an NGF-binding ELISA as described above. ATPA were detected using a bridging immunoassay. The administered pre-treatments are designated on the individual graph. The data show that in the cohort of animals given the recombinant mAb as a pre-treatment, no ATPA response was observed (FIG. 4F) while strong, sustained expression of feNGF mAb was observed in the serum (FIG. 4B). This suggests the rate of ATPA in cats in which intramuscular AAV.CB7.feNGF was administered is attenuated by pre-treatment with recombinant feNGF mAb protein, but not by other pre-treatments (FIGS. 4E, 4G, and 4H). Table 1 depicts a summary of the ATPA assessments observed.

TABLE 1

| | | Day(s) of | AAV-feNGF | ATPA |
|---|---|---|---|---|
| Group | Pre-treatment | pre-treatment | (IM) | Positive |
| 1 | None | — | AAV1-CB7-feNGF | 10/12 (83%) |
| 2 | Oral | daily from | AAV1-CB7-feNGF | 3/4 (75%) |
| | Prednisolone | Day −7 | | |
| | (1 mg/kg) | to Day 14 | | |
| 3 | feNGF mAb | Day-14 | AAV1-CB7-feNGF | 0/4 (0%) |
| | (2 mg/kg ) | | | |
| 4 | AAV-TBG-feNGF | Day-21 | AAV1-CB7-feNGF | 1/4 (25%) |
| | (IV) | | | |
| 5 | AAV-TBG-feNGF | Day-21 | AAV1-CB7-feNGF | 1/4 (25%) |
| | (IV) | | | |

The data demonstrate that pre-treatment with a recombinant protein of interest abrogates the immune response directed to that protein when it is delivered by AAV, resulting in long-term transgene persistence and expression. The results contained herein can readily be extended to other vectors types, vectors comprising other polynucleotides, and various species of subject using well known methods.

### Example 3: Animal Study of Anti-NGF mAb Followed by AAV Containing the Anti-NGF mAb as Compared to Anti-NGF mAb Alone for the Treatment of Pain in Cats with Degenerative Joint Disease (DJD)

Described herein is a placebo-controlled, 4-arm, partial cross-over, randomized, masked (double-blind) study comparing treatment with anti-NGF mAb to treatment with placebo, and comparing placebo to anti-NGF mAb followed by AAV containing the anti-NGF mAb (at two doses), for control of pain associated with degenerative joint disease (DJD, also called osteoarthritis (OA) in cats. The study includes placebo treatment for the control.

The objectives are:
1) To evaluate the effectiveness and field safety of anti-NGF mAb administered as a subcutaneous monthly injection for control of pain associated with OA in cats.
2) To evaluate the effectiveness and field safety of a single subcutaneous injection of anti-NGF mAb followed 13-15 days later by a single intramuscular (IM) injection of the AAV containing the anti-NGF mAb for control of pain associated with osteoarthritis (OA) in cats.

Enrollment criteria consists of cats under an established Veterinary Client Patient Relationship (VCPR) that have been diagnosed with OA by physical examination and radiography.

All study participant cats are treated with anti-NGF mAb at a point in the study. All study personnel are masked to treatment or placebo cohort. Day 0 is defined as the first day of dosing. Three groups of a minimum 33 cats are enrolled randomly in a 1:1:1 ratio.

Group 1 (MP) receives one subcutaneous injection of the anti-NGF mAb on Day 0, a placebo IM injection on Day 14, a subcutaneous injection of the anti-NGF mAb on Day and placebo subcutaneous injections on Days 90 and 120.

Group 2 (PM) receives one subcutaneous injection of placebo on Day 0, a placebo IM injection on Day 14, a placebo subcutaneous injection on Day 30, a subcutaneous injection of the anti-NGF mAb subcutaneous on Day 90 and Day 120.

Group 3 (MAL) receives the anti-NGF mAb as a subcutaneous injection on Day 0, the Low Dose (1×10¹² gc/cat) of AAV containing the anti-NGF mAb IM injection on Day 14, and placebo subcutaneous injections on Days 30, 90, and 120.

Group 4 (MAH) receives the anti-NGF mAb as a subcutaneous injection on Day 0, the High Dose (5×10¹² gc/cat) AAV containing the anti-NGF mAb IM injection on Day 14, and placebo SQ injections on Days 30, 90, and 120.

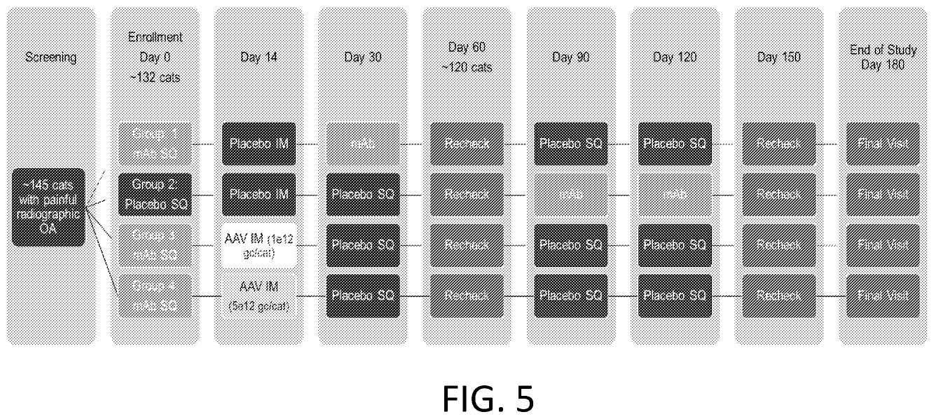

As shown in the study design diagram in FIG. 5, the study participant cats are evaluated at a screening visit (Day −14 to −3) and at visits on Days 0, 14, 30, 60, 90, 120, 150 and 180. The Day 14 visit occurs within ±1 day (i.e. a three-day window, Day 13-15) of the scheduled date, the Day 30 visit and all other visits occur within ±3 days.

Effectiveness of treatments is measured by comparison of assessments of Client Specific Outcome Measures [CSOM], Feline Musculoskeletal Pain Index-short form [FMPI-sf© ], Global Assessment. Assessments are performed at each visit during the study (Days 0, 30, 60, 90, 120, 150 and 180), except Day 14.

Safety of treatments is assessed through clinical pathology and recording of adverse events throughout the study.

Study participant cats treated with anti-NGF at Day 0 and Day 30 have increased success rates on Days 30, 60 and 90 as compared to cats treated with placebo at Day 0 and Day 30 (Group 1 (MP) vs. Group 2 (PM)).

Study participant cats treated with anti-NGF at Day 90 and Day 120 have increased success rates, on Days 120, 150 and 180, as compared to cats treated with placebo at Day and 120, (Group 2 (PM) vs. Group 1 (MP)).

Study participant cats treated with anti-NGF mAb and AAV containing the anti-NGF mAb have increased success rates on Days 30, 60 and 90 as compared to cats treated with placebo on study Days 0 and 30 (Group 3 (MAL) vs. Group 2 (PM); and Group 4 (MAH) vs. Group 2 (PM)).

Study participant cats treated with anti-NGF mAb and AAV containing the anti-NGF mAb have increased success rates, on Days 120, 150 and 180, as compared to cats treated with placebo on study Days 90 and 120 (Group 3 (MAL) vs. Group 1 (MP); and Group 4 (MAH) vs. Group 1 (MP)).

Within an individual animal, Client Specific Outcome Measures (CSOM) success rate increased 60 days after the first treatment when each animal is treated with 2 injections of anti-NGF as compared to when each animal is treated with 2 injections of placebo (analysis of cross-over in Groups 1 (MP) and 2 (PM)).

### Example 4: Attenuation of ATPA Response to a GLP-1 Analogue Following IM AAV Administration in Cats

This study evaluates the effect of pre-treatment of animals on the immunogenicity of the transgene product of a recombinant AAV that encodes a feline glucagon-like peptide-1 agonist (GLP-1) biologic drug consisting of a dipeptidyl peptidase-IV-protected GLP-1 analogue linked to feline serum IgG-Fc by a small peptide linker (fGLP-1mut-Fc).

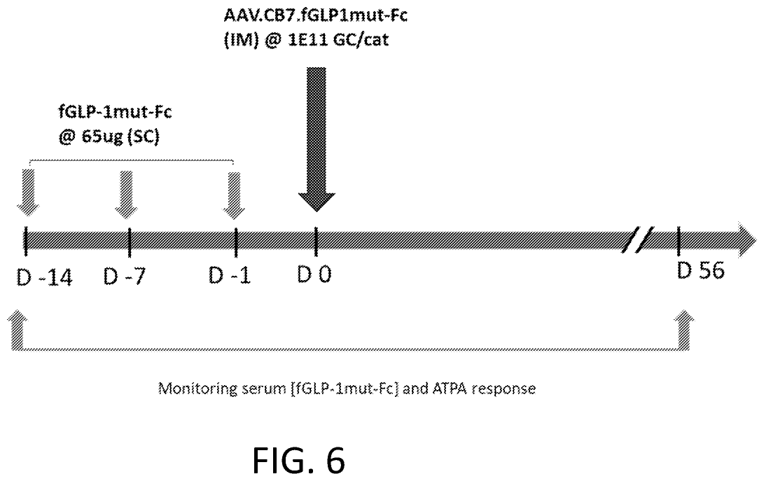

Adult cats are enrolled into one of two cohorts (n=8/cohort). As seen in FIG. 6, the first cohort receives a single IM dose of an AAV vector containing the transgene encoding the fGLP-1mut-Fc protein (AAV-fGLP-1mut-Fc). The vector is administered at 1×10¹¹ gc/cat on Day 0. The second cohort is given three subcutaneous doses, at 65 μg/cat, of recombinant purified feGLP-1mut-Fc at Day −14, Day −7 and Day −1 prior to also receiving the same AAV-fGLP-1mut-Fc dose as cohort 1 at Day 0.

Plasma levels of fGLP-1mut-Fc and anti-fGLP-1mut-Fc antibody responses are assessed at weekly intervals for the duration of the study using methodology outlined herein.

Quantification of fGLP-1Mut-Fc in Cat Plasma

The concentration of fGLP-1mut-Fc in cat plasma is determined using a high sensitivity GLP-1 active ELISA kit (#EZGLPHS-35K, Millipore). Briefly, pre-coated wells are incubated for 2 h at room temperature with plasma diluted in DPBS whilst shaking. Recombinant purified fGLP-1mut-Fc concentrations ranging from 50,000 pM to 0.8 pM are used to establish a standard curve. Additional steps are carried out as described in the kit instructions.

### Quantification of Anti-fGLP-1mut-Fc Antibodies in Cat Plasma

A bridging immunoassay with acid dissociation is used to evaluate the presence of anti-transgene product antibodies [ATPA, ie anti-(anti-fGLP-1mut-Fc)] antibodies in the cat plasma. Samples are pre-treated with acid to disrupt any existing ATPA— fGLP-1mut-Fc complexes before assessment in the bridging assay. Briefly, samples are diluted to 2.5% in 300 mM acetic acid to enable ATPA-drug complex dissociation before analysis. Acidified samples are incubated for 40 min with shaking at ambient temperature. 50 μl of the acidified samples are then transferred to wells of a 96-well plate containing 90 μL of master-mix reagent (0.063 μg/mL of biotinylated fGLP-1mut-Fc and 0.063 μg/mL of ruthenylated fGLP-1 mut-Fc with 1% Meso Scale Discovery (MSD) Blocker A in PBS-T) and 11 μL 1.2M Tris solution (pH 9.5). Acidified samples plus master-mix reagents are incubated at ambient temperature in the dark for 60 min with shaking. Streptavidin-coated MSD plates are blocked for 60 min at ambient temperature with 150 μL/well of PBS-T buffer containing 3% (MSD) Blocker A.

The Streptavidin-coated MSD plates are then washed and 50 μl of the acidified sample plus master-mix reagent are transferred to the plates which are incubated at ambient temperature in the dark for 60-90 min with shaking. The MSD plates are washed, 150 μL of 2×MSD Read T-Buffer is added to each well before the plates are read on an MSD MESO QuickPlex SQ 120 instrument. The resulting signal is recorded as Relative Light Units (RLU).

### Example 5: Attenuation of ATPA Response to Anti-IL-31 Following IM AAV Administration in Dogs

This study evaluates the effect of pre-treatment of animals on the immunogenicity of the transgene product of a recombinant AAV that encodes a canine anti-IL-31 antibody.

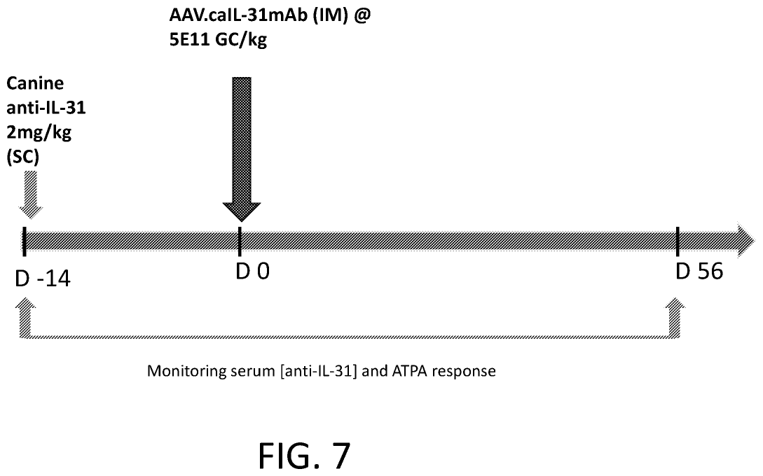

Adult dogs will be enrolled into one of two cohorts (n=6/cohort). As seen in FIG. 7, the first cohort receives a single IM dose of an AAV vector containing the transgene encoding the heavy and light chains of an IL-31 antibody (AAV-caIL-31). This is administered at 5×10¹¹ gc/kg on Day 0. The second cohort is given a 2 mg/kg subcutaneous dose of recombinant canine anti-IL-31 antibody at Day −14, prior to also receiving the same AAV-caIL-31 dose as cohort 1 at Day 0.

Serum levels of anti-IL-31 and anti-(anti caIL-31 antibody) antibody responses are assessed at weekly intervals for the duration of the study using methodology outlined herein.

### Quantification of Canine Anti-IL-31 Antibody in Dog Serum

The concentration of canine anti-IL-31 mAbs in dog serum is determined using an IL-31-binding ELISA. Briefly, ELISA plates are coated with 0.1 μg/ml caIL-31 and blocked with PBS/0.05% Tween 20/1% BSA. Coated wells are incubated for 1 h at room temperature with serum diluted in PBS/0.05% Tween 20/1% BSA (100 μl/well). Antibody concentrations ranging from 100 ng/ml to 1.56 ng/ml are used to establish a binding curve. After washing, the plates are incubated with a 1/10,000 dilution of goat anti-canine IgG-HRP in PBS/0.05% Tween 20/1% BSA. Plates are washed with PBS/0.05% Tween 20 and developed by the addition of TMB substrate. Development is stopped by the addition of 2M H₂SO₄ and absorbance is read at 450 nm and background is subtracted.

### Quantification of Anti-(Anti-IL-31 Antibody) Antibodies in Dog Serum

A bridging Immunoassay with acid dissociation is used to evaluate the presence of anti-transgene product antibodies [ATPA, ie anti-(anti-IL-31 antibody)] antibodies in the dog serum. Samples are pre-treated with acid to disrupt any existing ATPA— transgene/drug complexes before assessment in the bridging assay. Briefly, samples are diluted to 2.5% in 300 mM acetic acid to enable ATPA-drug complex dissociation before analysis. Acidified samples are incubated for 40 min with shaking at ambient temperature. 50 μl of the acidified samples are then transferred to wells of a 96 well plate containing 90 μL of master-mix reagent (0.063 μg/mL of biotinylated anti-IL-31 and 0.063 μg/mL of ruthenylated anti-IL-31 with 1% Meso Scale Discovery (MSD) Blocker A in PBS-T) and 11 μL 1.2 M Tris solution (pH 9.5). Acidified samples plus master-mix reagents are incubated at ambient temperature in the dark for 60 min with shaking. Streptavidin-coated MSD plates are blocked for 60 min at ambient temperature with 150 μL/well of PBS-T buffer containing 3% (MSD) Blocker A.

The Streptavidin-coated MSD plates are then washed and 50 μl of the acidified sample plus master-mix reagent are transferred to the plates which are incubated at ambient temperature in the dark for 60-90 min with shaking. The MSD plates are washed, 150 μL of 2×MSD Read T-Buffer is added to each well before the plates are read on an MSD MESO QuickPlex SQ 120 instrument. The resulting signal is recorded as Relative Light Units (RLU)

### Example 6: Attenuation of ATPA Response to Anti-NGF mAb Following IM AAV Administration in Dogs

This study evaluates the effect of pre-treatment of animals on the immunogenicity of the transgene product of a recombinant AAV that encodes a canine anti-NGF antibody.

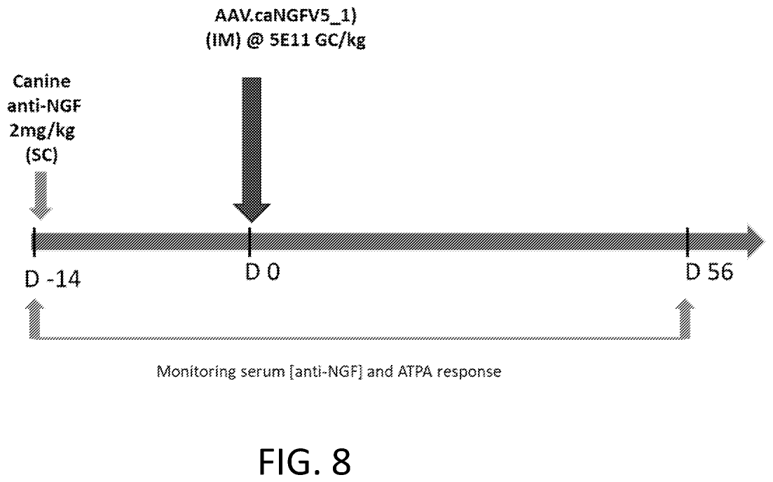

Adult dogs are enrolled into one of two cohorts (n=6/cohort). A seen in FIG. 8, the first cohort receives a single IM dose of an AAV vector containing the transgene encoding the heavy and light chains of an anti-canine NGF antibody (AAV-caNGF). This is administered at 5×10¹¹ gc/kg on Day 0. The second cohort will be given a 2 mg/kg subcutaneous dose of recombinant canine anti-NGF antibody at Day −14, prior to also receiving the same AAV-caNGF dose as cohort 1 at Day 0.

Serum levels of anti-NGF and anti-(anti-caNGF antibody) antibody responses are assessed at weekly intervals for the duration of the study using methodology outlined below.

### Quantification of Canine Anti-NGF Antibody in Dog Serum

The concentration of canine anti-NGF mAbs in dog serum is determined using an NGF-binding ELISA. Briefly, ELISA plates are coated with 0.1 μg/ml caNGF and blocked with PBS/0.05% Tween 20/1% BSA. Coated wells are incubated for 1 h at room temperature with serum diluted in PBS/0.05% Tween 20/1% BSA (100 μl/well). Antibody concentrations ranging from 100 ng/ml to 1.56 ng/ml are used to establish a binding curve. After washing, the plates are incubated with a 1/10,000 dilution of goat anti-canine IgG-HRP in PBS/0.05% Tween 20/1% BSA. Plates are washed with PBS/0.05% Tween 20 and developed by the addition of TMB substrate. Development is stopped by the addition of 2M H₂SO₄ and absorbance is read at 450 nm and background is subtracted.

### Quantification of Anti-(Anti-NGF Antibody) Antibodies in Dog Serum

A bridging Immunoassay with acid dissociation is used to evaluate the presence of anti-transgene product antibodies [ATPA, ie anti-(anti-NGF antibody)] antibodies in the dog serum. Samples are pre-treated with acid to disrupt any existing ATPA— transgene/drug complexes before assessment in the bridging assay. Briefly, samples are diluted to 2.5% in 300 mM acetic acid to enable ATPA-drug complex dissociation before analysis. Acidified samples are incubated for 40 min with shaking at ambient temperature. 50 μl of the acidified samples are then transferred to wells of a 96 well plate containing 90 μL of master-mix reagent (0.063 μg/mL of biotinylated anti-NGF and 0.063 μg/mL of ruthenylated anti-NGF with 1% Meso Scale Discovery (MSD) Blocker A in PBS-T) and 11 μL 1.2M Tris solution (pH 9.5). Acidified samples plus master-mix reagents are incubated at ambient temperature in the dark for 60 min with shaking. Streptavidin-coated MSD plates are blocked for 60 min at ambient temperature with 150 μL/well of PBS-T buffer containing 3% (MSD) Blocker A.

The Streptavidin-coated MSD plates are then washed and 50 μl of the acidified sample plus master-mix reagent are transferred to the plates which are incubated at ambient temperature in the dark for 60-90 min with shaking. The MSD plates are washed, 150 μL of 2×MSD Read T-Buffer is added to each well before the plates are read on an MSD MESO QuickPlex SQ 120 instrument. The resulting signal is recorded as Relative Light Units (RLU).

### Example 7: Attenuation of ATPA Response to Anti-NGF mAb Following IM AAV Administration in Cats

This example shows the attenuation of ATPA responses following pre-treatment of animals with two closely related antibodies, 14 days prior to AAV treatment cats were administered a single subcutaneous dose of either:
purified feNGF mAb (clone JCV4, SEQ ID NOs: 1 and 2) at a dose of 2 mg/kg
purified feNGF mAb (clone V5_1, SEQ ID NOs: 2 and 5) at a dose of 2 mg/kg

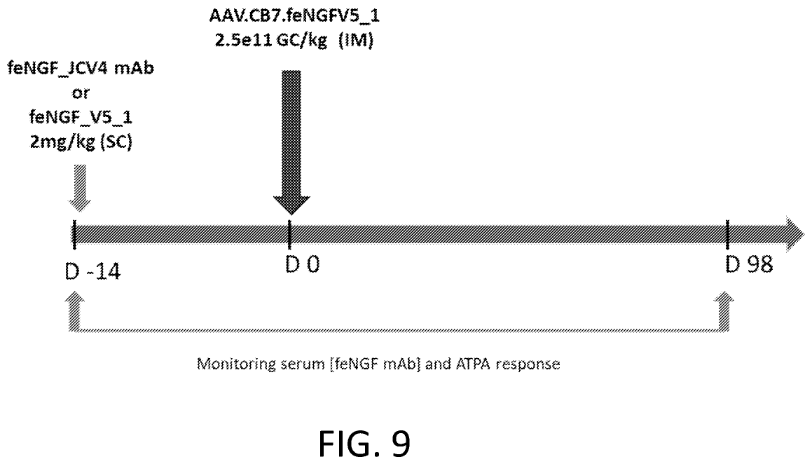

As seen in FIG. 9, cat subjects were then administered AAV containing a feNGF mAb transgene under the control of a CB7 promoter (not tissue specific) IM at a dose of 2.5×10¹¹ gc/kg at Day 0. A separate cohort of animals did not receive any pre-treatment prior to the AAV administration.

Serum levels of feNGF mAb were determined using an NGF-binding ELISA and ATPA were detected using a bridging immunoassay as detailed herein.

### Quantification of Feline Anti-NGF Antibody in Cat Serum

The concentration of feline anti-NGF mAb in cat serum was determined using an NGF-binding ELISA. Briefly, ELISA plates were coated with 0.1 μg/ml muNGF and blocked with PBS/0.05% Tween 20/1% BSA. muNGF coated wells were incubated for 1 h at room temperature with serum diluted in PBS/0.05% Tween 20/1% BSA (100 μl/well). Antibody concentrations ranging from 100 ng/ml to 1.56 ng/ml were used to establish a binding curve. After washing, the plates were incubated with a 1/10,000 dilution of goat anti-feline IgG-HRP in PBS/0.05% Tween 20/1% BSA. Plates were washed with PBS/0.05% Tween 20 and developed by the addition of TMB substrate. Development was stopped by the addition of 2M H₂SO₄ and absorbance read at 450 nm and background was subtracted.

### Assessment of Anti-Drug Antibodies (ADA) in Cat Serum

A bridging immunoassay with acid dissociation was used to evaluate the presence of anti-(anti-feNGF mAb) antibodies in the cat serum at timepoints when the circulating levels of drug were at the lowest (pre-dose). Samples were pre-treated with acid to disrupt any existing ADA-feNGF mAb complexes before assessment in the bridging assay. Briefly, samples were diluted to 2.5% in 300 mM acetic acid to enable ADA-drug complex dissociation before analysis. Acidified samples were incubated for 40 min with shaking at ambient temperature. 25 μl of the acidified samples were transferred to wells of a 96 well plate containing 90 μL of master-mix reagent (0.125 μg/mL of biotinylated feNGF mAb and 0.125 μg/mL of ruthenylated feNGF mAb with 1% Meso Scale Discovery (MSD) Blocker A in PBS-T) and 11 μL 1.2M Tris solution (pH 9.5). Acidified samples plus master-mix reagents were incubated at ambient temperature in the dark for 60 min with shaking. Simultaneously, Streptavidin-coated MSD plates were blocked for 60 min at ambient temperature with 200 μL/well of PBS-T buffer containing 3% (MSD) Blocker A.

The Streptavidin-coated MSD plates were then washed and 25 ul of the acidified sample plus master-mix reagent were transferred to the plates which were then incubated at ambient temperature in the dark for 60-90 min with shaking. The MSD plates were washed, 150 μL of 2×MSD Read T-Buffer was added per well before the plates were read on an MSD MESO QuickPlex SQ 120 instrument. The resulting response was recorded as Relative Light Units (RLU).

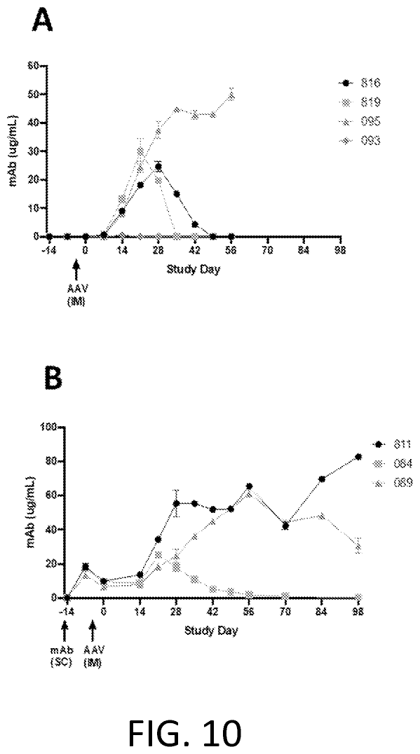

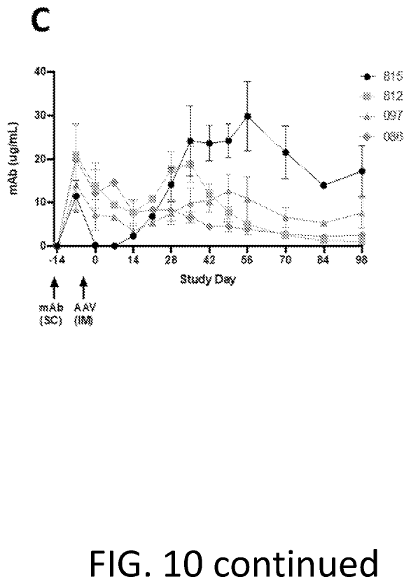

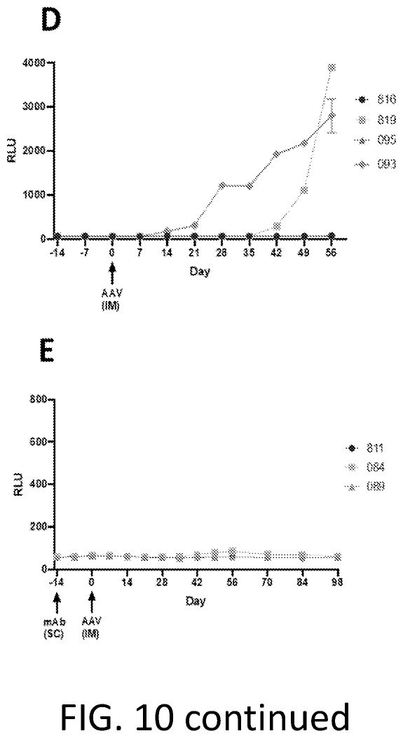

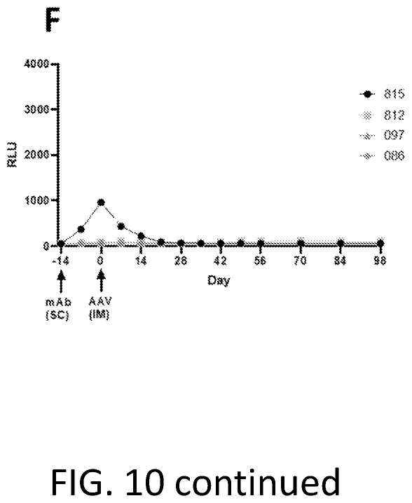

As seen in FIGS. 10A-10F, the high rate of ATPA in cats receiving AAV.CB7.feNGF IM is attenuated by pre-treatment with recombinant feNGF mAb protein.

Cats were administered various pre-treatments as listed above. AAV containing the feNGF mAb transgene was given at Day 0 at a dose of 2.5×10¹¹ gc/kg. In animals that received no pre-treatment (FIG. 10A and FIG. 10D) a high (50%) rate of ATPA was observed. In animals that received either the purified feNGF mAb (clone JCV4) (FIG. 10B and FIG. 10E) or purified feNGF mAb (clone V5_1) (FIG. 10C and FIG. 10F) no evidence of ATPA was observed up to 98 days post AAV administration. A low, transient ATPA signal was observed in one animal in the V5_1 pre-treatment group (FIG. 10F) following the recombinant mAb administration that did not persist and did not affect expression of the transgene following AAV administration.

These data show that the ATPA response to AAV-delivered antibody can be attenuated by administration of a recombinant version of the antibody or by a closely related antibody.

### Example 8: Attenuation of ATPA Response to Single Pre-Treatment Anti-NGF mAb Following IM AAV Administration in Cats

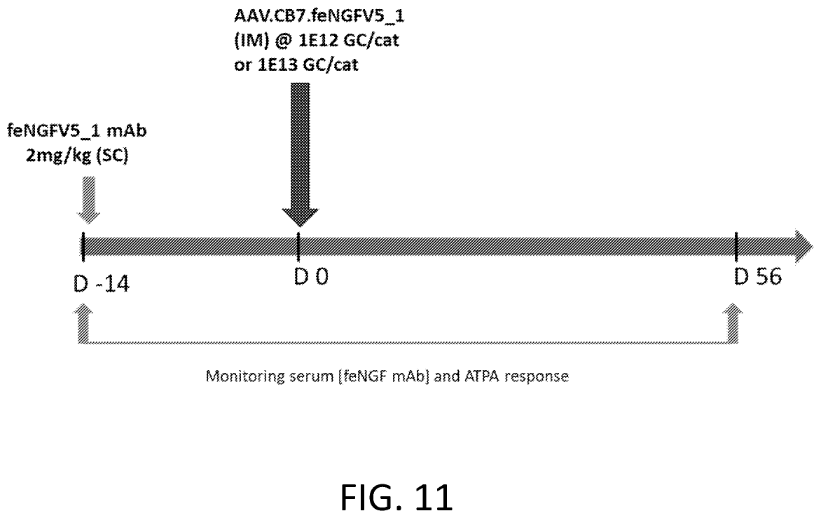

This example compares the effect of a single antibody pre-treatment on the attenuation of ATPA generated by AAV administered products at a medium and high dose. As seen in FIG. 11, 14 days prior to AAV treatment, cat subjects were administered a single subcutaneous dose of purified feNGF mAb (clone V5_1, SEQ ID NOs: 2 and 5) at a dose of 2 mg/kg. Cats were then administered AAV containing a feNGF mAb transgene (feNGFV5_1) at two different doses: either 1×10¹² gc/cat or 1×10¹³ gc/cat at Day 0. A separate cohort of animals did not receive any pre-treatment prior to the AAV administration. A group size of 8 animals/cohort was used for this study.

Serum levels of feNGF mAb were determined using an NGF-binding ELISA and ATPA were detected using a bridging immunoassay as detailed herein.

### Quantification of Feline Anti-NGF Antibody in Cat Serum

The concentration of feline anti-NGF mAbs in cat serum was determined using an NGF-binding ELISA. Briefly, ELISA plates were coated with 0.1 μg/ml muNGF and blocked with PBS/0.05% Tween 20/1% BSA. muNGF coated wells were incubated for 1 h at room temperature with serum diluted in PBS/0.05% Tween 20/1% BSA (100 μl/well). Antibody concentrations ranging from 100 ng/ml to 1.56 ng/ml were used to establish a binding curve. After washing, the plates were incubated with a 1/10,000 dilution of goat anti-feline IgG-HRP in PBS/0.05% Tween 20/1% BSA. Plates were washed with PBS/0.05% Tween 20 and developed by the addition of TMB substrate. Development was stopped by the addition of 2M H₂SO₄ and absorbance read at 450 nm and background was subtracted.

### Assessment of Anti-Drug Antibodies (ADA) in Cat Serum

A bridging immunoassay with acid dissociation was used to evaluate the presence of anti-(anti-feNGF mAb) antibodies in the cat serum at timepoints when the circulating levels of drug were at the lowest (pre-dose). Samples were pre-treated with acid to disrupt any existing ADA-feNGF mAb complexes before assessment in the bridging assay. Briefly, samples were diluted to 2.5% in 300 mM acetic acid to enable ADA-drug complex dissociation before analysis. Acidified samples were incubated for 40 min with shaking at ambient temperature. 2511.1 of the acidified samples were transferred to wells of a 96 well plate containing 90 μL of master-mix reagent (0.125 μg/mL of biotinylated feNGF mAb and 0.125 μg/mL of ruthenylated feNGF mAb with 1% Meso Scale Discovery (MSD) Blocker A in PBS-T and 11 μL 1.2 M Tris solution (pH 9.5). Acidified samples plus master-mix reagents were incubated at ambient temperature in the dark for 60 min with shaking. Simultaneously, streptavidin-coated MSD plates were blocked for 60 min at ambient temperature with 200 μL/well of PBS-T buffer containing 3% (MSD) Blocker A.

The streptavidin-coated MSD plates were then washed and 25 μl of the acidified sample plus master-mix reagent were transferred to the plates which were then incubated at ambient temperature in the dark for 60-90 min with shaking. The MSD plates were washed, 150 μL of 2×MSD Read T-Buffer was added per well before the plates were read on an MSD MESO QuickPlex SQ 120 instrument. The resulting response was recorded as Relative Light Units (RLU).

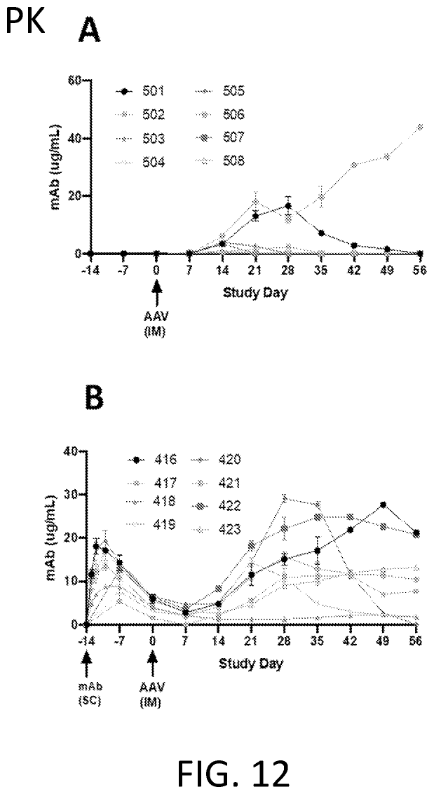

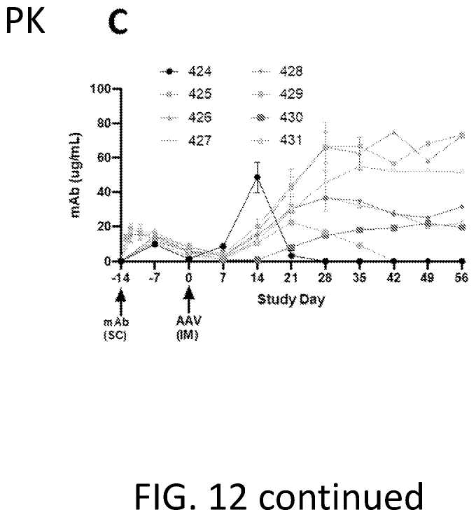

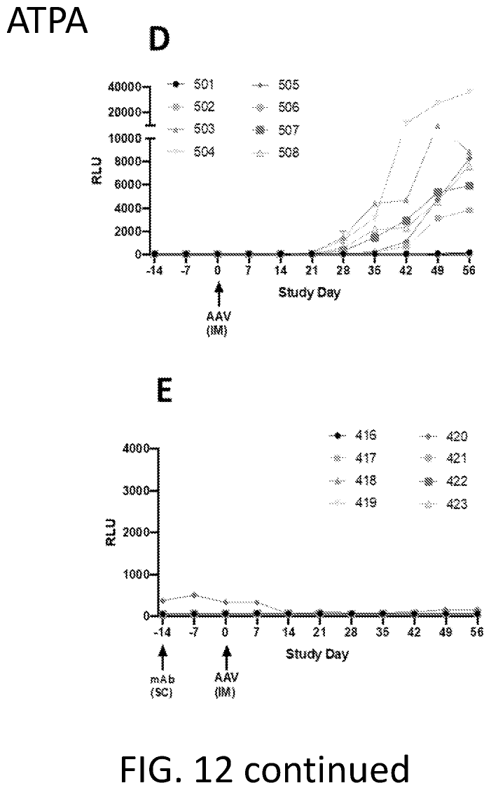

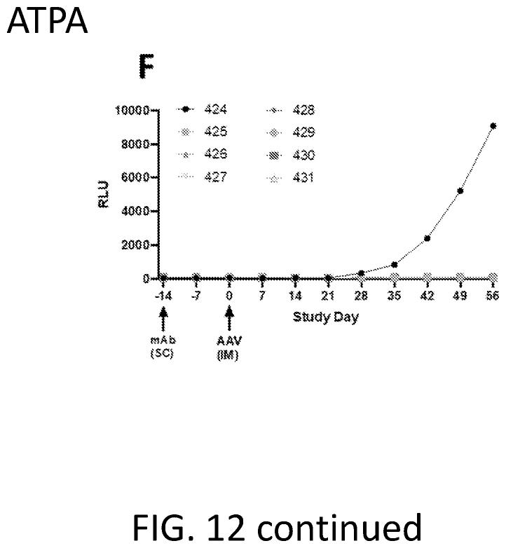

As seen in FIGS. 12A-12F, a single subcutaneous dose of recombinant antibody attenuates the ATPA response generated by a single dose of AAV-delivered antibody at two different gc/kg dose levels.

Cats were administered a single subcutaneous dose of purified feNGF mAb (clone V5_1) 14 days prior to receiving either a medium (FIG. 12B and FIG. 12E) or high (FIG. 12C and FIG. 12F) dose of an AAV containing the feNGF mAb (clone V5_1) transgene at Day 0. In animals that received no pre-treatment (FIG. 12A and FIG. 12D) a high (50%) rate of ATPA was observed at Day 56.

In animals that received the medium dose of AAV (1×10¹² gc/cat) (FIG. 12B and FIG. 12E) no evidence of treatment-emergent immunogenicity was observed up to 56 days post AAV administration. A low, transient reactivity was observed in one animal prior to treatment with antibody at Day −14. This did not appear to affect the pharmacokinetics (PK) of the recombinant mAb nor the subsequent expression of antibody from the AAV vector. A single treatment induced ATPA response was observed in the high dose (1×10¹³ gc/cat) cohort (FIG. 12C and FIG. 12F) that correlated with a sudden loss in detectable serum antibody.

These data show that the ATPA response to AAV-delivered antibody can be attenuated by administration of a recombinant version of the antibody, even when the AAV dose is considered high.

A summary of the ATPA attenuation data is seen in Table 2.

TABLE 2

| | | Treatment-Induced |
|---|---|---|
| mAb Pre-Treatment | AAV Treatment | ATPA at Day 56 |
| — | AAV1-fNGF_JCV4 | 10/12 cats (83%) |
| fNGF_JCV4 mAb | AAV1-fNGF_JCV4 | 0/4 cats (0%) |
| — | AAVrh91-fNGFV5_1 | 2/4 cats (50%) |
| fNGF_JCV4 mAb | AAVrh91-fNGFV5_1 | 0/3 cats (0%) |
| fNGFV5_1 mAb | AAVrh91-fNGFV5_1 | 0/4 cats (0%) |
| — | AAVrh91-fNGFV5_1 | 6/8 (75%) |
| fNGFV5_1 mAb | AAVrh91-fNGFV5_1 | 1/16 (6.25%) |

1. A method for suppressing an immune response to a therapeutic protein encoded by a vector in a subject in need thereof, for expressing a therapeutic protein in a subject in need thereof, and/or for treating a disease or disorder associated with reduced expression of a therapeutic protein in a subject in need thereof, the method comprising:
a) selecting a subject as the subject in need of treatment with the vector, and then
b) administering to the subject a composition comprising the therapeutic protein or a homolog or functional variant thereof, optionally an immunologically related variant thereof.

2. The method of claim 1, wherein the method comprises administering to the subject the vector comprising the polynucleotide encoding the therapeutic protein or a homolog or functional variant thereof, optionally an immunologically related variant thereof.

3. The method of claim 1, wherein the method comprises administering to the subject the composition comprising the therapeutic protein or a homolog or functional variant thereof, optionally an immunologically related variant thereof, and administering to the subject the vector comprising the polynucleotide encoding the therapeutic protein or a homolog or functional variant thereof, optionally an immunologically related variant thereof.

4. The method of any one of claims 1-3, wherein the therapeutic protein or a homolog or functional variant thereof, optionally an immunologically related variant thereof is administered before the vector is administered.

5. The method of any one of claims 1-4, wherein the therapeutic protein or a homolog or functional variant thereof, optionally an immunologically related variant thereof is administered after the vector is administered.

6. The method of any one of claims 1-5, wherein the therapeutic protein or a homolog or functional variant thereof, optionally an immunologically related variant thereof is administered at about the same time the vector is administered.

7. The method of any one of claims 1-6, wherein the vector is a viral vector.

8. The method of claim 7, wherein the viral vector is an adeno-associated virus (AAV) vector.

9. The method of any one of claims 1-8, wherein the therapeutic protein or a homolog or functional variant thereof, optionally an immunologically related variant thereof is administered intramuscularly.

10. The method of any one of claims 1-9, wherein the therapeutic protein or a homolog or functional variant thereof, optionally an immunologically related variant thereof is administered intravenously.

11. The method of any one of claims 1-10, wherein the therapeutic protein or a homolog or functional variant thereof, optionally an immunologically related variant thereof or a functional fragment of the therapeutic protein or a homolog or functional variant thereof, optionally an immunologically related variant thereof shares at least 95% identity to an endogenous protein of the subject or a functional fragment of the endogenous protein.

12. The method of any one of claims 1-11, wherein the therapeutic protein or a homolog or functional variant thereof, optionally an immunologically related variant thereof is recognized by the immune system as a self-antigen.

13. The method of any one of claims 1-12, wherein the composition is administered to the subject at a dose effective to suppress an immune response to the therapeutic protein or a homolog or functional variant thereof, optionally an immunologically related variant thereof encoded by the vector.

14. The method of any one of claims 1-13, wherein the therapeutic protein or a homolog or functional variant thereof, optionally an immunologically related variant thereof comprises an antibody or antigen-binding fragment thereof.

15. The method of claim 14, wherein the therapeutic protein or a homolog or functional variant thereof, optionally an immunologically related variant thereof is a monoclonal antibody.

16. The method of claim 15, wherein the monoclonal antibody is a monoclonal antibody that specifically binds Nerve Growth Factor (NGF).

17. The method of claim 15, wherein the monoclonal antibody is a monoclonal antibody that specifically binds EPO, IL-31, VEGF, CD20, Her 2, TNF, IL-2, IgE, IL-33, CD52, CD3, CD19, IL-6, IL-4, IL-4R, IL-13, IL-13R, IL-5, IL-5R, IL-33R, α4β7 integrin, IL-12, IL-23, GMCSF, GMCSFR, PD-1, PD-L1, CTLA-4, B7-1, or B7-2.

18. The method of any one of claims 1-13, wherein the therapeutic protein or a homolog or functional variant thereof, optionally an immunologically related variant thereof is GLP-1, insulin, GnRH, or MIS/AMH.

19. The method of any one of claims 1-13, wherein the therapeutic protein or a homolog or functional variant thereof, optionally an immunologically related variant thereof is an agonist or antagonist of GLP-1 insulin, GnRH, or MIS/AMH.

20. The method of any one of claims 1-19, wherein the subject is a feline.

21. The method of any one of claims 1-19, wherein the subject is a canine.

22. The method of any one of claims 1-19, wherein the subject is a human.

23. The method of claim 20, wherein the therapeutic protein or a homolog or functional variant thereof, optionally an immunologically related variant thereof is a feline monoclonal antibody or a felinized monoclonal antibody.

24. The method of any one of claims 1-23, therein the subject is a juvenile or an adult.

25. The method of any one of claims 1-24, wherein the therapeutic protein or a homolog or functional variant thereof, optionally an immunologically related variant thereof is administered at a dose of less than about 50 mg/kg, less than about 25 mg/kg, less than about 10 mg/kg, less than about 5 mg/kg, less than about 1 mg/kg, or less than about 0.5 mg/kg.

26. The method of any one of claims 1-25, wherein the therapeutic protein or a homolog or functional variant thereof, optionally an immunologically related variant thereof is administered at a dose of at least about 0.01 mg/kg, at least about 0.05 mg.kg, at least about 0.1 mg/kg, at least about 1 mg/kg, at least about 2 mg/kg, at least about 5 mg/kg, at least about 10 mg/kg, or at least about 15 mg/kg.

27. The method of any one of claims 1-26, wherein the therapeutic protein or a homolog or functional variant thereof, optionally an immunologically related variant thereof is administered at a dose of about 0.1 mg/kg to about 50 mg/kg, about 1 mg/kg to about 50 mg/kg, about 0.1 mg/kg to about 25 mg/kg, about 1 mg/kg to about 25 mg/kg, or about 5 mg/kg to about 10 mg/kg.

28. The method of any one of claims 1-26, wherein the therapeutic protein or a homolog or functional variant thereof, optionally an immunologically related variant thereof is administered at a dose of about 0.1 mg/kg, about 1 mg/kg, about 2 mg/kg, about 5 mg/kg, about 10 mg/kg, or about 15 mg/kg.

29. The method of any one of claims 1-28, wherein the therapeutic protein or a homolog or functional variant thereof, optionally an immunologically related variant thereof is administered at a sub-therapeutic dose for the therapeutic protein or a homolog or functional variant thereof, optionally an immunologically related variant thereof.

30. The method of any one of claims 1-29, wherein the therapeutic protein or a homolog or functional variant thereof, optionally an immunologically related variant thereof is administered to the subject for less than 6 weeks, less than 5 weeks, less than 4 weeks, less than 3 weeks, less than 2 weeks, less than 1 week, less than 5 days, less than 3 days, or less than one day before the vector is administered.

31. The method of any one of claims 1-30, wherein the immune response is an antibody-based immune response of the subject to the therapeutic protein or a homolog or functional variant thereof, optionally an immunologically related variant thereof in response to expression of the therapeutic protein or a homolog or functional variant thereof, optionally an immunologically related variant thereof by cells transduced with the vector in the subject.

32. The method of any one of claims 1-31, wherein the method reduces the host antibody response by at least about 20%.

33. The method of any one of claims 1-31, wherein the method reduces the host antibody response by at least about 50%.

34. The method of any one of claims 1-31, wherein the method reduces the host antibody response by at least about 75%.

35. The method of any one of claims 1-31, wherein the method reduces the host antibody response by at least about 90%.

36. The method of claim 8, wherein the AAV vector is an AAV1 vector.

37. The method of claim 8, wherein the AAV vector is an AAVrh91 vector.

38. The method of any one of claims 1-37, wherein the vector is administered at a dose of 1×10⁷ to 1×10¹⁵ gc/kg.

39. The method of any one of claims 1-37, wherein the vector is administered at a dose of 1×10⁹ to 1×10¹³ gc/kg.

40. The method of any one of claims 1-37, wherein the vector is administered at a dose of 2.5×10″ gc/kg.

41. The method of any one of claims 1-40, wherein the therapeutic protein or a homolog or functional variant thereof, optionally an immunologically related variant thereof is administered 1 day after, 2 days after, 5 days after, 7 days after, 10 days after, or 20 days after the vector is administered.

42. The method of any one of claims 1-41, wherein the subject is treatment-naïve to the therapeutic protein or a homolog or functional variant thereof, optionally an immunologically related variant thereof.

43. The method of any one of claims 1-42, wherein the therapeutic protein or a homolog or functional variant thereof, optionally an immunologically related variant thereof is administered at a time which would allow measurable levels of the therapeutic protein or a homolog or functional variant thereof, optionally an immunologically related variant thereof to be present at the time of administration of the vector.

44. The method of any one of claims 1-43, wherein the therapeutic protein or a homolog or functional variant thereof, optionally an immunologically related variant thereof is administered before expression of the therapeutic protein or a homolog or functional variant thereof, optionally an immunologically related variant thereof by the vector would be expected.

45. The method of any one of claims 1-44, wherein the disease or disorder is degenerative joint disease (DJD).
